(19) United States
(12) Patent Application Publication
(10) Pub. No.: US 2023/0045390 A1
(43) Pub. Date: Feb. 9, 2023

(54) PLASMODIUM VIVAX MALE GAMETE FUSION PROTEIN PVHAP2 AND THE PUTATIVE PROLIFERATING-CELL NUCLEOLAR ANTIGEN P120 AS RELAPSE BIOMARKERS

(71) Applicant: Seattle Children's Hospital dba Seattle Children's Research Institute, Seattle, WA (US)

(72) Inventors: Stefan H.I. Kappe, Seattle, WA (US); Noah Sather, Seattle, WA (US); Gigliola Zanghi, Seattle, WA (US); Erika L. Flannery, Oakland, CA (US); Hemando Del Portillo, Barcelona (ES); Melisa Gualdron Lopez, Barcelona (ES); Carmen Fernández-Becerra, Barcelona (ES)

(73) Assignee: Seattle Children's Hospital dba Seattle Children's Research Institute, Seattle, WA (US)

(21) Appl. No.: 17/810,982

(22) Filed: Jul. 6, 2022

Related U.S. Application Data
(60) Provisional application No. 63/218,635, filed on Jul. 6, 2021.

(51) Int. Cl. G01N 33/569; C07K 14/445; C07K 16/20; G01N 33/68

(52) U.S. Cl. CPC G01N 33/56905; C07K 14/445; C07K 16/205; G01N 33/6848; G01N 2333/445; A61K 2039/505

(57) ABSTRACT

The present disclosure provides a method of detecting the presence of a hypnozoite stage of Plasmodium vivax in a liver cell. In addition, the present disclosure provides compositions and methods of detecting the presence of a latent Plasmodium vivax infection in a subject and for treating the subject detected to be infected.

### CROSS-REFERENCE TO RELATED APPLICATION

This application claims the benefit of U.S. Patent Application No. 63/218,635, filed Jul. 6, 2021, expressly incorporated herein by reference in its entirety.

### STATEMENT OF GOVERNMENT LICENSE RIGHTS

This invention was made with government support under AI135680 awarded by the National Institutes of Health. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3399-P33US_SEQ_final_2022-7-5. The XML file is 8 KB; was created on Jul. 5, 2022; and is being submitted via Patent Center with the filing of the specification.

### BACKGROUND

Plasmodium vivax is the most widely distributed malaria parasite responsible for the majority of malaria cases outside sub-Saharan Africa with 2.5 billion people at risk. This parasite caused 7.5 million malaria cases last year distributed in Central and Southeast Asia, Americas and Eastern parts of Africa. Although P. vivax causes less mortality than P. falciparum, severe disease and complicated malaria are also attributable exclusively to this species. The biology of P. vivax is complex and differs in several aspects from P. falciparum making it difficult to eliminate P. vivax infections with similar control and treatment strategies. After transmission of infectious sporozoite stages by mosquito bite, P. vivax is characterized by the formation of latent liver stage forms derived from some sporozoites, called hypnozoites. These non-replicating forms can persist in the human liver for months to years and then reactivate to undergo schizogony and produce infectious merozoite forms that initiate recurrent and clinically manifest blood infections called relapses⁴. Numerous studies indicate that relapses are responsible for up to 79% of all P. vivax malaria cases in several endemic countries. The presence of hypnozoites in the human population thus constitute a major challenge to the World Health Organization malaria eradication goal. Individuals harboring silent hypnozoites are not only suffering recurrent blood stage infections, but they are also the source for continued parasite transmission.

In recent years P. vivax hypnozoite biology has started to be unveiled, owing to the development of new technologies. The construction of a humanized liver chimeric FRG huHepmouse in vivo model for liver stages, together with optimized (PHH) in vitro culture systems, including micropatterned cocultures and, more recently liver spheroids, have allowed the uncovering of unknown aspects of hypnozoites. Thus, transcriptional analysis of in vitro cultured P. vivax, and simian P. cynomolgi hypnozoites has revealed gene expression patterns indicating that mature hypnozoites have a reduced transcriptional activity. Yet, dormant hypnozoites express genes involved in energy metabolism, transcriptional and translational control, protein export, quiescence, and maintenance of genome integrity. Moreover, P. vivax infection in FRG huHep mice has shown that hypnozoites perform active cellular processes such us endoplasmic reticulum biogenesis as well as apicoplast and mitochondrial replication. More recently, the creation of a dual reporter P. cynomolgi cell line has allowed the observation of individual hypnozoites transitioning to replicating schizonts, a major breakthrough in hypnozoite biology and a unique platform for the screening of putative anti-relapse drugs.

Relapses are effectively prevented by 8-aminoquinoline drugs including Primaquine (PQ) and more recently Tafenoquine (TQ), presumably by directly eliminating hypnozoites in the liver. However, because hypnozoite infection cannot be diagnosed, WHO recommends their administration in combination with drugs (Chloroquine) against blood stages of schizonts for P. vivax radical cure treatment. Yet, the mass implementation of such treatments has strong limitations due to the acute hemolytic anemia occurring in patients with glucose-6-phosphate dehydrogenase deficiency (G6PDd) when exposed to 8-aminoquinolines and the risk of taking these drugs for pregnant women.

Accordingly, despite the advances in the art, a need remains to reliably detect the presence of hypnozoites in carriers to reliably guide therapeutic intervention to eradicate latent liver stage infections with P. vivax and avoid relapses and reduce transmission of the parasite.

### SUMMARY

In accordance with the foregoing, the present disclosure provides a method of detecting the presence of a hypnozoite stage of Plasmodium vivax in a liver cell. The method of this aspect comprises:

obtaining one or more extracellular vesicles (EVs) secreted by the liver cell; and

detecting the presence of male gamete membrane fusion protein (HAP2) protein and/or proliferating cell nucleolar antigen P120 (putative; PVP01_0930100) in the one or more EVs.

The presence of HAP2 protein and/or proliferating cell nucleolar antigen P120 in the one or more EVs is indicative of the presence of the hypnozoite stage of P. vivax. Thus, HAP2 protein and/or proliferating cell nucleolar antigen P120 is detected in the one or more EVs when the hypnozoite stage of P. vivax is present in the liver cell.

In some embodiments, the step of obtaining one or more EVs comprises performing size exclusion chromatography (SEC) on a sample containing EVs. Obtaining one or more EVs can further comprise detecting an EV marker to confirm the presence of EVs in the sample. Exemplary, non-limiting examples of EV markers can include CD9, CD81, and/or CD5L. Detection of such EV markers can be performed with reagents and according to techniques known in the art.

In some embodiments, detecting the presence of HAP2 protein or proliferating cell nucleolar antigen P120 comprises contacting the one or more EVs with an affinity reagent that binds to HAP2 or proliferating cell nucleolar antigen P120, respectively.

In some embodiments, the affinity reagent is detectably labeled. A label or detectable label, as used herein, refers to a moiety attached to an affinity reagent that permits observation or detection of the affinity reagent. A label can produce a signal itself that is detectable by visual or instrumental approaches. Various labels include signal-producing substances, luminescent moieties, bioluminescent moieties, radioactive moieties, positron emitting metals, nonradioactive paramagnetic metal ions, and the like.

In some embodiments, the step of detecting the presence of HAP2 protein and/or proliferating cell nucleolar antigen P120 comprises digesting proteins in EVs followed by, for example, liquid chromatograph (LC) and mass spectrometry (MS) according to standard techniques.

The liver cell being assessed can be cultured in vitro or, alternatively, can be in vivo in a subject with a suspected latent infection of P. vivax. In cases where the cell is in vivo, the step of obtaining one or more EVs can comprise obtaining a liquid biological sample from a subject with a suspected latent infection of P. vivax. In some embodiments, the liquid biological sample is or comprises blood or plasma.

In some embodiments, the subject is human.

In another aspect, the disclosure provides a method of detecting the presence of a latent Plasmodium vivax infection in a subject. The method of this aspect comprises:

obtaining a liquid biological sample from the subject comprising one or more extracellular vesicles (EVs) secreted by liver cells; and

detecting the presence of male gamete membrane fusion protein (HAP2) protein and/or proliferating cell nucleolar antigen P120 (putative; PVP01_0930100) in the one or more EVs.

HAP2 protein and/or proliferating cell nucleolar antigen P120 is detected in the one or more EVs when the subject has a latent P. vivax infection. In this regard, the latent P. vivax can be characterized by the presence of a hypnozoite stage of P. vivax in a liver cell in the subject.

In some embodiments, the method further comprises isolating one or more EVs from the liquid biological sample, such as performing size exclusion chromatography (SEC) on the liquid biological sample, as described above and below. This isolation can further comprise detecting an EV marker to confirm the presence of EVs in the sample. Exemplary EV markers include CD9, CD81, and CD5L.

In some embodiments, the method comprises detecting the presence of HAP2 protein and/or proliferating cell nucleolar antigen P120 by contacting the one or more EVs with an affinity reagent that specifically binds to HAP2 or proliferating cell nucleolar antigen P120, respectively. Exemplary affinity reagents are described above. In some embodiments, the affinity reagents are detectably labeled, as described above.

In other embodiments, the step of detecting the presence of HAP2 protein or proliferating cell nucleolar antigen P120 comprises digesting proteins in EVs followed by liquid chromatograph (LC) and mass spectrometry (MS), as described above.

The liquid biological sample can be or comprise blood or plasma.

In some embodiments, if the subject is determined to have a latent P. vivax infection, the method further comprises treating the subject for P. vivax. Exemplary treatments can comprise administering to the subject a therapeutic amount of a composition comprising 8-aminoquinoline. For example, a composition comprising 8-aminoquinoline can be Primaquine or Tafenoquine.

In some embodiments, the subject is human.

In yet another aspect, the disclosure provides a method of diagnosing and treating a subject with a latent Plasmodium vivax infection, comprising:

obtaining a biological sample from the subject with one or more extracellular vesicles (EVs) secreted by the subject's liver cells;

detecting the presence of male gamete membrane fusion protein (HAP2) protein and/or proliferating cell nucleolar antigen P120 (putative; PVP01_0930100) in the one or more EVs, wherein a detected presence of the HAP2 protein and/or the proliferating cell nucleolar antigen P120 in the one or more EVs indicates a latent infection of P. vivax in the subject; and treating the latent P. vivax infection in the subject.

In some embodiments, the latent P. vivax is characterized by the presence of a hypnozoite stage of P. vivax in a liver cell in the subject. The biological sample can be a liquid biological sample.

In some embodiments, the method further comprises isolating one or more EVs from the liquid biological sample, such as performing size exclusion chromatography (SEC) on the liquid biological sample, as described herein above and below. In some embodiments, the method further comprises detecting an EV marker to confirm the presence of EVs in the biological sample. Exemplary EV markers include CD9, CD81, and/or CD5L.

In some embodiments, the step of detecting the presence of HAP2 protein or proliferating cell nucleolar antigen P120 comprises contacting the one or more EVs with an affinity reagent that specifically binds to HAP2 or proliferating cell nucleolar antigen P120, respectively. Exemplary affinity reagents are described above. In some embodiments, the affinity reagents are detectably labeled, as described above.

In other embodiments, the step of detecting the presence of HAP2 protein or proliferating cell nucleolar antigen P120 comprises digesting proteins in EVs followed by liquid chromatograph (LC) and mass spectrometry (MS), as described above.

The liquid biological sample can be or comprise blood or plasma.

In some embodiments, the method of treating can comprise administering to the subject a therapeutic amount of a composition comprising 8-aminoquinoline.

In some embodiments, if the subject is determined to have a latent P. vivax infection, the method further comprises treating the subject for P. vivax. Exemplary treatments can comprise administering to the subject a therapeutic amount of a composition comprising 8-aminoquinoline. For example, a composition comprising 8-aminoquinoline can be Primaquine or Tafenoquine.

In some embodiments, the subject is human.

### DESCRIPTION OF THE DRAWINGS

This patent or application file contains at least one drawing executed in color. Copies of this patent or patent application publication with color drawing(s) will be provided by the Office upon request and payment of the necessary fee.

The foregoing aspects and many of the attendant advantages of this invention will become more readily appreciated as the same become better understood by reference to the following detailed description, when taken in conjunction with the accompanying drawings, wherein:

FIGS. 1A-1D. Identification of P. vivax hypnozoite biomarkers in the in vitro model of P. vivax liver infection. Extraerythrocytic forms (EEF) quantification of P. vivax PHH-infected in vitro cultures from total EEF (FIG. 1A) and schizontes/hypnozoites ratio (FIG. 1B). Quantifications were obtained by IFA manually counting small and large UIS4/HSP70 positively stained parasites. Data represent mean and standard error of measurements performed in four wells. Statistical significance of total EEF quantification was assessed by unpaired t-test (***P<0.001) and Schizont/hypnozoite number by Two-way ANOVA Sidak's multiple comparison test (***<0.001). FIG. 1C. Table showing number of H. sapiens and P. vivax proteins identified in EVs from P. vivax infected and MMV048-treated PHH. FIG. 1D. Venn diagram showing the comparison of H. sapiens proteins identified in EVs from vivax infected PHH and EVs from previous in vitro culture of PRH, HepAD38 HBV and Huh-7 HBV cell lines. Gene names of 12 common proteins are listed.

FIGS. 2A-2D. Characterization of EVs from plasma of P. vivax infected FRG huHep mice. FIG. 2A. Schematic representation of experimental groups of P. vivax infected FRG huHEP mice treated with MMV048. Three mice were used per treatment in each group. Group 1 corresponds to uninfected FRG huHep mice. Group 2 corresponds to mice infected by mosquito bite and treated at day 4 post-infection with MMV048 intravenously or DMSO as control. Animals from this group were euthanized at 8 days post infection (dpi). Group 3 and 4 correspond to mice intravenously infected with P. vivax sporozoites and treated at 4 days (Group 3) and 17 days (Group 4) post-infection with MMV048 intravenously or DMSO as control. Mice from these two groups were euthanized at 8 and 21 days respectively. Group 5 corresponds to mice intravenously infected with P. vivax sporozoites and treated at 14 dpi with Tafenoquine intravenously. Mice from this group were euthanized at 21 dpi. FIG. 2B. Characterization of P. vivax liver infection in FRG huHep mice treated with MMV048. Representative image of IFA analysis showing an hypnozoite form in the liver of MMV048-treated mice from group 4 (top) and a schizont form in the DMSO-treated control mice from group 3 (bottom). Scale bar in top=5 μm and in bottom=10 μm. FIG. 2C. Quantification of liver stages by IFA. FIG. 2D. Quantification of parasite load by RT-qPCR (log 10 Plasmodium 18S rRNA per μg Liver RNA). Data represent mean and standard error of measurements perform in individual mice. Statistical significance was assessed by paired t-test *P<0.05, **P<0.01.

FIGS. 3A-3D. Identification of P. vivax hypnozoite biomarkers associated to plasma-derived EVs in the FRG huHep mice in vivo model. FIG. 3A. Total proteins identified from H. sapiens, M musculus and P. vivax in plasma derived EVs from all experimental groups. FIG. 3B. Venn diagram showing comparison of human proteins identified in FRG huHep mice from this study. FIG. 3C. Number of P. vivax proteins identified in plasma derived EVs from each experimental group. Data represent mean and standard error of P. vivax proteins identified in mice from each group. FIG. 3D. Distribution P. vivax proteins in different subcellular compartment as predicted by GO enrichment terms and Uniprot cell compartment assignations. Membranes, cytosol, nucleus, and undetermined compartments are represented. Red asterisk (*) refers to proteins with no orthologues in P. falciparum.

FIGS. 4A-4E. Statistical analysis of human proteome of plasma-derived EVs from P. vivax infected FRG huHep mice. Human proteins associated to plasma derived EVs from P. vivax infected FRG huHep mice were compared between different experimental groups as indicated in statistical analysis of material and methods. Images show human proteins in which it was found statistically significant association to infection (FIG. 4A); Hypnozoite infection (FIG. 4B); Tafenoquine treatment (FIG. 4C); infection mode (4D); and infection time (8 days vs. 21 days) (FIG. 4E). Estimate refers to fold change between conditions. (P<0.05).

FIG. 5. Schematic representation of the experimental set up of P. vivax in vitro infection and MMV048 treatment of PHH. PHH were infected with P. vivax sporozoites. 72 hours post-infection infected cultures were treated with MMV048 (0.1 μM) and continue cultured up to 144 hours post-infection. Conditioned medium was collected daily for EVs isolation.

FIGS. 6A and 6B. Molecular characterization of PHH-derived EVs by flow cytometry. EVs from culture supernatant of P. vivax-infected PHH were purified by SEC. FIG. 6A. SEC fractions were analyzed for the presence of CD9 and CD81 EVs marker in a flow cytometry bead-based assay (BBA). Negative controls refer to fractions F7-F8-conjugated beads incubated with an isotype antibody and Alexa Flour™ 488 secondary antibody or fractions F8-F9-conjugated beads incubated with Alexa Flour™ 488 secondary antibody. FIG. 6B. CD9⁺CD81⁺ single pick SEC fractions from all conditions were further tested for CD63 and MHCI by BBA. Negative control refers to CD9⁺CD81⁺ single pick SEC fraction conjugated beads incubated with Alexa Flour™ 488 secondary antibody in the absence of primary antibodies. Cut-off values were determined as the mean plus two-fold standard deviation from all samples.

FIGS. 7A-7C. Nanoparticle track analysis of P. vivax infected PHH-derived EVs. Plots show size (mode (FIG. 7A) and mean (FIG. 7B)) and concentration (FIG. 7C) of particles from CD9⁺ pick SEC fraction from all experimental conditions. Data represent mean and standard error of three measurements performed in each sample. Statistically significant differences between groups were tested in a paired t-test. (*P<0.05; **P<0.01).

FIGS. 8A-8D. Cryo-TEM analysis of EVs enriched SEC fractions from P. vivax infected PHH. Images showing isolated EVs from culture supernatant of P. vivax infected PHH treated with DMSO (FIG. 8A) and MMV048 (FIG. 8B) after 120-144 hours post-infection. (FIG. 8C) EV diameters were quantified using ImageJ (NIH) where pixels were calibrated to nanometers. Mean diameter obtained after analysis of 12-15 pictures/condition is shown. (FIG. 8D) EV size distribution.

FIGS. 9A-9E. Molecular characterization of plasma derived EVs from P. vivax infected FRG huHep mice treated with MMV048 by flow cytometry. Plots show individual P. vivax infected FRG huHep mice SEC profiles. SEC fractions were analyzed for the presence of CD9 and CD5L EV markers in a flow cytometry bead-based assay. Negative control refers to fractions F6-F11-conjugated beads incubated with Alexa Flour™ 488 secondary antibody. (FIG. 9A) Experimental group 1: Uninfected mice; (FIG. 9B) Experimental group 2: Mosquito-bite infected mice at 8 dpi; (FIG. 9C) Experimental group 3: Intravenous infected mice at 8 dpi; (FIG. 9D) Experimental group 4: Intravenous infected mice at 21 dpi and (FIG. 9E) Intravenous infected mice treated with Tafenoquine at 21 dpi.

FIG. 10. Molecular analysis of additional EV markers in CD9⁺CD5L⁺ single pick SEC fractions from plasma-derived EVs from P. vivax infected FRG huHEP mice. CD9⁺CD5L⁺ single pick SEC fractions from individual mice were further tested for CD63, CD81 and HLA-I by BBA. Negative control refers to CD9+CD5L⁺ single pick SEC fraction conjugated beads incubated with Alexa Flour™ 488 secondary antibody in the absence of primary antibodies. Cut-off values were determined as the mean plus two-fold standard deviation from all samples.

FIGS. 11A-11C. Nanoparticle track analysis of plasma-derived EVs from P. vivax infected FRG huHEP mice. Plots show size (mode (FIG. 11A) and mean (FIG. 11B)) and concentration (FIG. 11C) of particles from CD5L/CD9⁺ pick SEC fractions from all experimental groups. Data represent mean and standard error of individual measurements performed in each mouse. Statistically significant differences between groups were tested in a paired t-test. No differences were found.

FIGS. 12A-12D. Cryo-TEM analysis of plasma-derived EVs from P. vivax infected FRG huHEP mice. Images showing isolated EVs from plasma of P. vivax infected FRG huHEP mice by mosquito-bite and treated with DMSO (FIG. 12A) or MMV048 (FIG. 12B). EVs diameter were quantified using ImageJ (NIH) where pixels were calibrated to nanometers (FIG. 12C). Mean diameter obtained after analysis of 12-13 pictures/condition is shown. FIG. 12D. EV size distribution.

FIG. 13. Sequence analysis of C-terminal region of Plasmodium spp male gamete fusion protein HAP2. Male game Fusion protein HAP2 protein sequences from Plasmodium spp were aligned using Clustal Omega. PfDd2_100019600-t41_1-p1, PfGA01_100019500-t41_1-p1, PfIT_100018200-t41_1-p1, PfKE01_100019500-t41_1-p1, PfH01_100018800-t41_1-p1, PfKH02_100019700-t41_1-p1, PfML01_100018500-t41_1-p1, Pf3D7_1014200.1-p1, PfCD01_100019400-t41_1-p1, PfGN01_100019800-t41_1-p1, Pf5D01_100018900-t41_1-p1, Pf5N01_100019600-t41_1-p1, PfTG01_100019400-t41_1-p1, Pf7G8_100018600-t411-p1, PfGB4_100019200-t41_1-p1, PfHB3_100018600-t41_1-p1 comprise the amino acid sequence of SEQ ID NO:1; PocGH01_08022600.1-p1 comprises the amino acid sequence of SEQ ID NO:2; PmUG01_08030200.1-p1 comprises the amino acid sequence of SEQ ID NO:3; PKNH_0814100.1-p1 and PKNOH_S100042100-t35_1-p1 comprise the amino acid sequence of SEQ ID NO:4; PVL_080018800-t42_1-p1 comprises the amino acid sequence of SEQ ID NO:5; PVP01_0814300.1-p1 and PVX_094925.′-p1 comprise the amino acid sequence of SEQ ID NO:6; and PcyM_0814900-t36_1-p1 comprises the amino acid sequence of SEQ ID NO:7. Pink areas indicate conserved amino acids in hypnozoite forming species (P. vivax SalI, P. vivax PVP01, P. vivax-like, P. knowlesi and P. cynomolgy). A 14 amino acid peptide is uniquely present in these species as compared to P. falciparum strains (black arrow).

FIG. 14. Abundance distribution of human proteins in plasma-derived EVs from P. vivax infected FRG huHEP mice. Plots shows that per sample log 10 protein abundance distribution usually lie within a similar range.

### DETAILED DESCRIPTION

As indicated above, latent livers stages termed “hypnozoites” cause relapsing Plasmodium vivax malaria infection and represent a major obstacle in the goal of malaria elimination. Hypnozoites are clinically undetectable and presently there are no biomarkers of this persistent parasite reservoir in the human liver. As described in more detail below, it is demonstrated herein that extracellular vesicles secreted from in vitro and in vivo infections exclusively containing hypnozoites, contain parasite proteins. Briefly, P. vivax infected primary human hepatocytes (PHH) and infected FRG huHep mice treated with the schizonticidal experimental drug MMV048 were used as hypnozoite infection models. Immunofluorescence-based quantification of P. vivax liver forms showed that MMV048 removed schizonts from infected PHH and livers from chimeric mice. Proteomic analysis of PHH-derived EVs and FRG huHep mice identified 7 and 66 P. vivax proteins, respectively. Remarkably, one protein from PHH cultures and six from FRG huHep mice were exclusively associated with hypnozoite infections. This study provides novel diagnostic tools for the identification of asymptomatic hypnozoite carriers in human populations and will facilitate precise intervention to treat latent infections and further reduce transmission rates.

In accordance with the foregoing, the present disclosure provides a method of detecting the presence of a hypnozoite stage of Plasmodium vivax in a liver cell. The method of this aspect comprises:

obtaining one or more extracellular vesicles (EVs) secreted by the liver cell; and detecting the presence of male gamete membrane fusion protein (HAP2) protein and/or proliferating cell nucleolar antigen P120 (putative; PVP01_0930100) in the one or more EVs.

The presence of HAP2 protein and/or proliferating cell nucleolar antigen P120 in the one or more EVs is indicative of the presence of the hypnozoite stage of P. vivax. Thus, HAP2 protein and/or proliferating cell nucleolar antigen P120 is detected in the one or more EVs when the hypnozoite stage of P. vivax is present in the liver cell.

In some embodiments, the step of obtaining one or more EVs comprises performing size exclusion chromatography (SEC) on a sample containing EVs. Obtaining one or more EVs can further comprise detecting an EV marker to confirm the presence of EVs in the sample. Exemplary, non-limiting examples of EV markers can include CD9, CD81, and/or CD5L. Detection of such EV markers can be performed with reagents and according to techniques known in the art.

In some embodiments, detecting the presence of HAP2 protein or proliferating cell nucleolar antigen P120 comprises contacting the one or more EVs with an affinity reagent that binds to HAP2 or proliferating cell nucleolar antigen P120, respectively.

The term “affinity reagent” refers to a molecule that can selectively bind to a desired antigen, e.g., HAP2 protein or proliferating cell nucleolar antigen P120 (putative; PVP01_0930100). A variety of affinity reagent formats and platforms are known and are encompassed by this disclosure. In some embodiments, the indicated affinity reagent can be an antibody or an antibody-like molecule such as an antigen-binding fragment or derivative thereof.

The term “antibody” refers to a polypeptide ligand that includes at least a light chain or heavy chain immunoglobulin variable region and specifically binds an epitope of an antigen, such as HAP2 protein or proliferating cell nucleolar antigen P120. The term “antibody” encompasses antibodies derived from any antibody-producing mammal (e.g., mouse, rat, rabbit, and primate including human) that specifically bind to the antigen of interest. Exemplary antibodies include multi-specific antibodies (e.g., bispecific antibodies); humanized antibodies; murine antibodies; chimeric, mouse-human, mouse-primate, primate-human monoclonal antibodies; and anti-idiotype antibodies.

The term “antibody-like molecule” includes functional fragments or derivatives of intact antibody molecules, such as molecules that comprise portions of an antibody, or modified antibody molecules. Typically, antibody-like molecules retain specific binding functionality, such as by retention of, e.g., with a functional antigen-binding domain of an intact antibody molecule. Preferably antibody fragments include the complementarity determining regions (CDRs), antigen binding regions, or variable regions thereof.

Illustrative examples of antibody fragments and derivatives useful in the present disclosure include Fab, Fab′, F(ab)₂, F(ab′)₂ and Fv fragments, nanobodies (e.g., VHH fragments and VNAR fragments), linear antibodies, single-chain antibody molecules, multi-specific antibodies formed from antibody fragments, and the like. Single-chain antibodies include single-chain variable fragments (scFv) and single-chain Fab fragments (scFab). A “single-chain Fv” or “scFv” antibody fragment, for example, comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Single-chain antibodies can also include diabodies, triabodies, and the like. Antibody fragments can be produced recombinantly, or through enzymatic digestion.

The above affinity reagent does not have to be naturally occurring or naturally derived, but can be further modified to, e.g., reduce the size of the domain or modify affinity for the antigen as necessary. For example, complementarity determining regions (CDRs) can be derived from one source organism and combined with other components of another, such as human, to produce a chimeric molecule that avoids stimulating immune responses in a subject.

Production of antibodies or antibody-like molecules can be accomplished using any technique commonly known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Once a monoclonal antibody is identified for inclusion within the bi-specific molecule, the encoding gene for the relevant binding domains can be cloned into an expression vector that also comprises nucleic acids encoding the remaining structure(s) of the bi-specific molecule.

Antibody fragments that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art.

Other affinity reagents encompassed by the disclosure include aptamers. As used herein, the term “aptamer” refers to oligonucleic or peptide molecules that can bind to specific antigens of interest. Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phosphodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer/target binding.

Peptide aptamers are protein molecules that often contain a peptide loop attached at both ends to a protamersein scaffold. The loop typically has between 10 and 20 amino acids long, and the scaffold is typically any protein that is soluble and compact. One example of the protein scaffold is Thioredoxin-A, wherein the loop structure can be inserted within the reducing active site. Peptide aptamers can be generated/selected from various types of libraries, such as phage display, mRNA display, ribosome display, bacterial display, and yeast display libraries.

The term “specifically binds” refers to, with respect to a target antigen, the preferential association of an antibody or other affinity reagent, in whole or part, with the target antigen, such as a transcription-associated histone modification or another affinity reagent. A specific affinity reagent binds substantially only to a defined target, such as a transcription-associated histone modification or another affinity reagent. It is recognized that a minor degree of non-specific interaction may occur between a molecule, such as a specific binding agent, and a non-target polypeptide. Nevertheless, specific binding can be distinguished as mediated through specific recognition of the antigen. Although selectively reactive affinity reagents, e.g., antibodies, bind antigen, they can do so with low affinity. Specific binding typically results in greater than 2-fold, such as greater than 5-fold, greater than 10-fold, or greater than 100-fold increase in amount of bound affinity reagent (per unit time) to a target antigen, such as compared to a non-target polypeptide. A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

In some embodiments, the affinity reagent is detectably labeled. A label or detectable label, as used herein, refers to a moiety attached to an affinity reagent that permits observation or detection of the affinity reagent. A label can produce a signal itself that is detectable by visual or instrumental approaches. Various labels include signal-producing substances, luminescent moieties, bioluminescent moieties, radioactive moieties, positron emitting metals, nonradioactive paramagnetic metal ions, and the like. A nonlimiting example of a luminescent moiety includes luminol; non-limiting examples of bioluminescent moieties include luciferase, luciferin, and aequorin; and nonlimiting examples of suitable radioactive moieties include a radioactive metal ion, e.g., alpha-emitters or other radioisotopes such as, for example, iodine (¹³¹I, ¹²⁵I, ¹²³I, ¹²¹I), carbon (¹⁴C), sulfur (³⁵S), tritium (³H), indium (^{115m}In, ^{113m}In, ¹¹²In, ¹¹⁴In), and technetium (⁹⁹Tc, ⁹⁹mTc), thallium (²⁰¹Ti), gallium (⁶⁸Ga, (WO2016/073853) ⁶⁷Ga), palladium (¹⁰³Pd), molybdenum (⁹⁹Mo), xenon (¹³³Xe), fluorine (¹⁰F), Samarium (¹⁴⁷Sm), Lu, ¹⁵⁹Gd, ¹⁴⁹Pm, ⁴⁰La, ⁷⁵Yb, ¹⁶⁶Ho, ⁹⁰Y, ⁴⁷Sc, ⁸⁶R, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ³⁴Mn, ⁷⁵Se, and tin (¹¹³Sn, ¹¹⁷Sn). The detectable moiety can be coupled or conjugated either directly to the affinity reagent of the disclosure or indirectly, through an intermediate (such as, for example, a linker) using suitable techniques.

A label can also be a moiety that does not itself emit a signal but can be detected upon its activity with a substrate. For example, the label can be a suitable enzyme, such as horseradish peroxidase, alkaline phosphatase, β-galactosidase, glucose oxidase, or acetylcholinesterase, that can facilitate a detectable signal under specifically applied conditions using known substrates. Again, the detectable moiety can be coupled or conjugated either directly or indirectly to the antibody or antibody derivatives of the disclosure.

In some embodiments, the step of detecting the presence of HAP2 protein and/or proliferating cell nucleolar antigen P120 comprises digesting proteins in EVs followed by, for example, liquid chromatograph (LC) and mass spectrometry (MS) according to standard techniques. See the disclosure below.

The liver cell being assessed can be cultured in vitro or, alternatively, can be in vivo in a subject with a suspected latent infection of P. vivax. In cases where the cell is in vivo, the step of obtaining one or more EVs can comprise obtaining a liquid biological sample from a subject with a suspected latent infection of P. vivax. In some embodiments, the liquid biological sample is or comprises blood or plasma.

In some embodiments, the subject is human.

In another aspect, the disclosure provides a method of detecting the presence of a latent Plasmodium vivax infection in a subject. The method of this aspect comprises:

obtaining a liquid biological sample from the subject comprising one or more extracellular vesicles (EVs) secreted by liver cells; and

detecting the presence of male gamete membrane fusion protein (HAP2) protein and/or proliferating cell nucleolar antigen P120 (putative; PVP01_0930100) in the one or more EVs.

HAP2 protein and/or proliferating cell nucleolar antigen P120 is detected in the one or more EVs when the subject has a latent P. vivax infection. In this regard, the latent P. vivax can be characterized by the presence of a hypnozoite stage of P. vivax in a liver cell in the subject.

In some embodiments, the method further comprises isolating one or more EVs from the liquid biological sample, such as performing size exclusion chromatography (SEC) on the liquid biological sample, as described above and below. This isolation can further comprise detecting an EV marker to confirm the presence of EVs in the sample. Exemplary EV markers include CD9, CD81, and CD5L.

In some embodiments, the method comprises detecting the presence of HAP2 protein and/or proliferating cell nucleolar antigen P120 by contacting the one or more EVs with an affinity reagent that specifically binds to HAP2 or proliferating cell nucleolar antigen P120, respectively. Exemplary affinity reagents are described above. In some embodiments, the affinity reagents are detectably labeled, as described above.

In other embodiments, the step of detecting the presence of HAP2 protein or proliferating cell nucleolar antigen P120 comprises digesting proteins in EVs followed by liquid chromatograph (LC) and mass spectrometry (MS), as described above.

The liquid biological sample can be or comprise blood or plasma.

In some embodiments, if the subject is determined to have a latent P. vivax infection, the method further comprises treating the subject for P. vivax. Exemplary treatments can comprise administering to the subject a therapeutic amount of a composition comprising 8-aminoquinoline. For example, a composition comprising 8-aminoquinoline can be Primaquine or Tafenoquine.

In some embodiments, the subject is human.

In yet another aspect, the disclosure provides a method of diagnosing and treating a subject with a latent Plasmodium vivax infection, comprising:

obtaining a biological sample from the subject with one or more extracellular vesicles (EVs) secreted by the subject's liver cells;

detecting the presence of male gamete membrane fusion protein (HAP2) protein and/or proliferating cell nucleolar antigen P120 (putative; PVP01_0930100) in the one or more EVs, wherein a detected presence of the HAP2 protein and/or the proliferating cell nucleolar antigen P120 in the one or more EVs indicates a latent infection of P. vivax in the subject; and

treating the latent P. vivax infection in the subject.

In some embodiments, the latent P. vivax is characterized by the presence of a hypnozoite stage of P. vivax in a liver cell in the subject. The biological sample can be a liquid biological sample.

In some embodiments, the method further comprises isolating one or more EVs from the liquid biological sample, such as performing size exclusion chromatography (SEC) on the liquid biological sample, as described herein above and below. In some embodiments, the method further comprises detecting an EV marker to confirm the presence of EVs in the biological sample. Exemplary EV markers include CD9, CD81, and/or CD5L.

In some embodiments, the step of detecting the presence of HAP2 protein or proliferating cell nucleolar antigen P120 comprises contacting the one or more EVs with an affinity reagent that specifically binds to HAP2 or proliferating cell nucleolar antigen P120, respectively. Exemplary affinity reagents are described above. In some embodiments, the affinity reagents are detectably labeled, as described above.

In other embodiments, the step of detecting the presence of HAP2 protein or proliferating cell nucleolar antigen P120 comprises digesting proteins in EVs followed by liquid chromatograph (LC) and mass spectrometry (MS), as described above.

The liquid biological sample can be or comprise blood or plasma.

In some embodiments, the method of treating can comprise administering to the subject a therapeutic amount of a composition comprising 8-aminoquinoline.

In some embodiments, if the subject is determined to have a latent P. vivax infection, the method further comprises treating the subject for P. vivax. Exemplary treatments can comprise administering to the subject a therapeutic amount of a composition comprising 8-aminoquinoline. For example, a composition comprising 8-aminoquinoline can be Primaquine or Tafenoquine.

In some embodiments, the subject is human.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, N.Y. (2001); Ausubel, F. M., et al. (eds.), Current Protocols in Molecular Biology, John Wiley & Sons, New York (2010); Bonifacino, J. S., et al. (eds), Current Protocols in Cell Biology, John Wiley & Sons, New York (1999); Phillips, M., Burrows, J., Manyando, C. et al. Malaria. Nat. Rev. Dis. Primers 3, 17050 (2017) (//doi.org/10.1038/nrdp.2017.50); and Mueller I, et al. Key gaps in the knowledge of Plasmodium vivax, a neglected human malaria parasite. Lancet Infect. Dis. 2009 September; 9(9):555-566, for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.” Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, and the like, of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention and are not intended to limit the scope of what the inventors regard as their invention nor are they intended to represent that the experiments below are all or the only experiments performed.

### Example 1

Plasmodium vivax constitutes one of the greatest obstacles to achieving the World Health Organization's target of eliminating malaria from 35 countries by 2030. This is because the parasite forms liver stages called hypnozoites that can remain latent in the liver during several years after the initial infection and upon reactivation cause disease relapse and perpetuate transmission. Until recently, there are no diagnostic tools capable of detecting asymptomatic hypnozoite carriers. It was previously demonstrated that extracellular multi-vesicles, nanovesicles of endocytic origin, obtained from the plasma of a humanized chimeric liver model infected with P. vivax, contain parasite proteins. This example describes additional work where hypnozoite-infected mice and hypnozoite-infected primary human liver cells were treated with a schizonticidal experimental drug to remove schizonts. After drug treatment, P. vivax proteins were identified. These results provide biomarkers to detect asymptomatic latent liver infections in P. vivax and, thus, contribute to malaria elimination.

Extracellular vesicles are double membrane nanovesicles secreted by all cell types and are involved in intercellular communication. These vesicles are originated from different cell compartments including multivesicular bodies (exosomes), plasma membrane (microvesicles, small vesicles, oncosomes, apoptotic bodies) and shows high heterogeneity in size and molecular composition. The molecular content of EVs (protein, lipids, nucleic acids, and metabolites) reflect the physiological status of the cell of origin. This feature, together with the fact that EVs are present in all biological fluids so far studied, has prompted its exploration as biomarkers in liquid biopsies of a wide range of pathologies including cancer, neurological disorders, diseases affecting lungs, kidney, liver, as well as infectious diseases.

Numerous studies have demonstrated that malaria infected cells secrete EVs that contain parasite proteins and are involved in host-parasite interactions. These include cell-cell communication, modulation of immune responses, alterations of vascular endothelium, cerebral malaria pathogenesis and induction of adhesion molecules in spleen fibroblast, among others. While most studies have been focused in vesicles derived from blood-stage parasites, it remains to be determined if EVs derived from hepatocytes infected with Plasmodium liver stages have any function in intercellular communication and can identify biomarkers of latent Plasmodium vivax liver infection. It has been previously demonstrated that plasma-derived EVs isolated from the liver stage model of P. vivax infected FRG huHep mice contain parasite proteins indicating the potential of this model for discovering hypnozoite biomarkers. However, an important limitation of our previous study was that livers of infected FRG huHep mice contained both replicating schizonts together with hypnozoites, precluding us from distinguishing EVs exclusively derived from hypnozoite-infected hepatocytes. Here, an experimental approach exploiting the schizonticidal properties of the experimental drug MMV048 has been employed to generate in vitro and in vivo infections of P. vivax hypnozoites to explore the protein content of EVs derived from hypnozoite-infected hepatocytes.

### Results

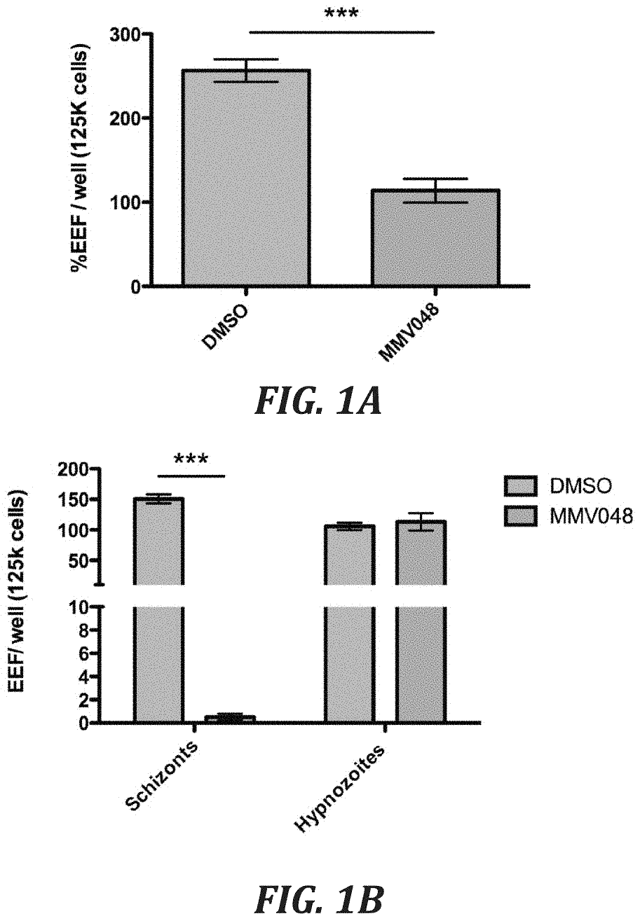

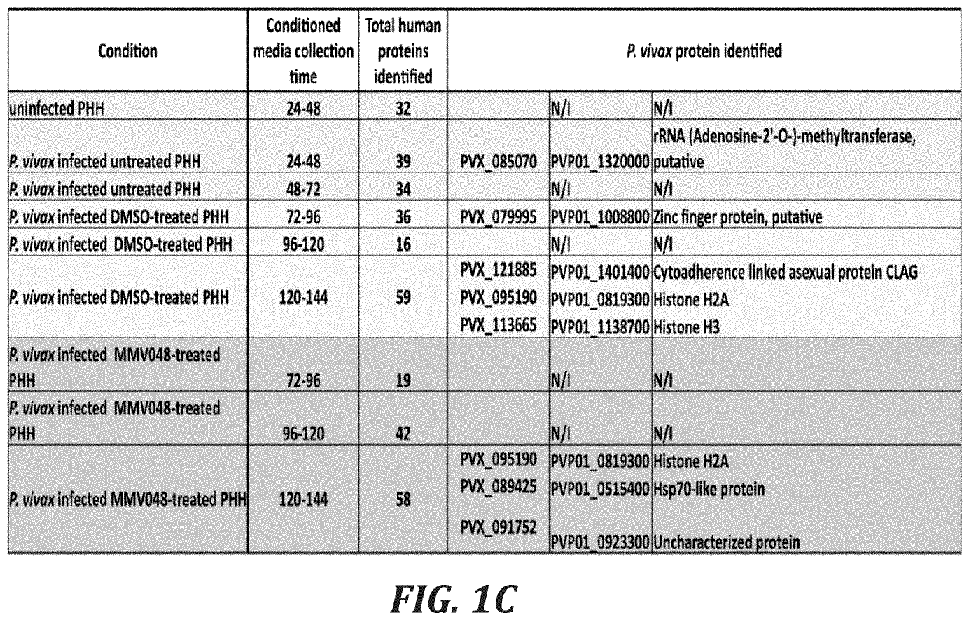

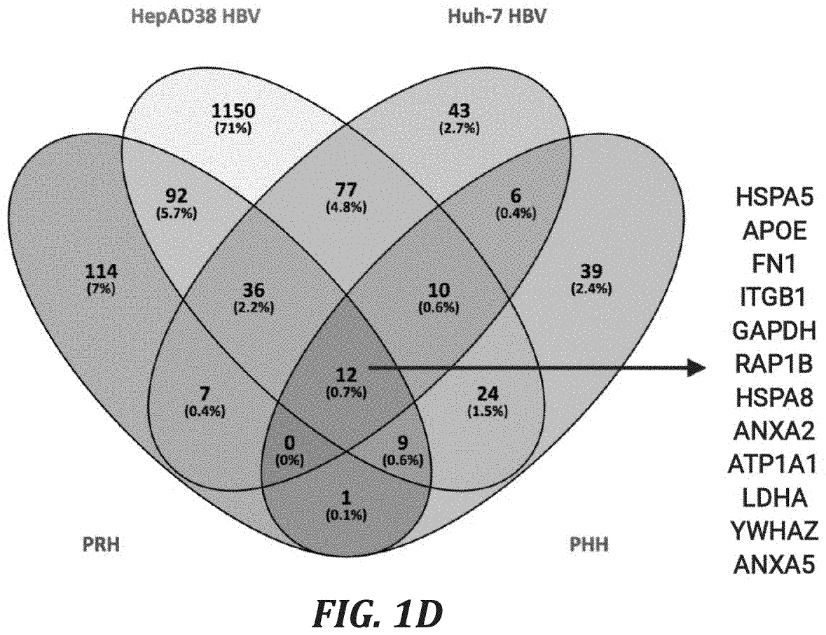

Identification of P. vivax hypnozoites biomarkers in the in vitro model of P. vivax liver infection PHH were infected in vitro with P. vivax sporozoites and treated with the schizonticidal experimental drug MMV048. Quantification of total extraerythrocytic forms (EEF) by IFA showed significant reduction in MMV048-treated cultures when compared to DMSO-treated cells (FIG. 1A). Noticeably, no schizonts were observed in MMV048- treated cells (only hypnozoites were seen) while in DMSO treatment there was a mix of hypnozoites and schizonts (FIG. 1B), demonstrating the schizonticidal effect of MMV048 and the resistance of hypnozoites to the drug.

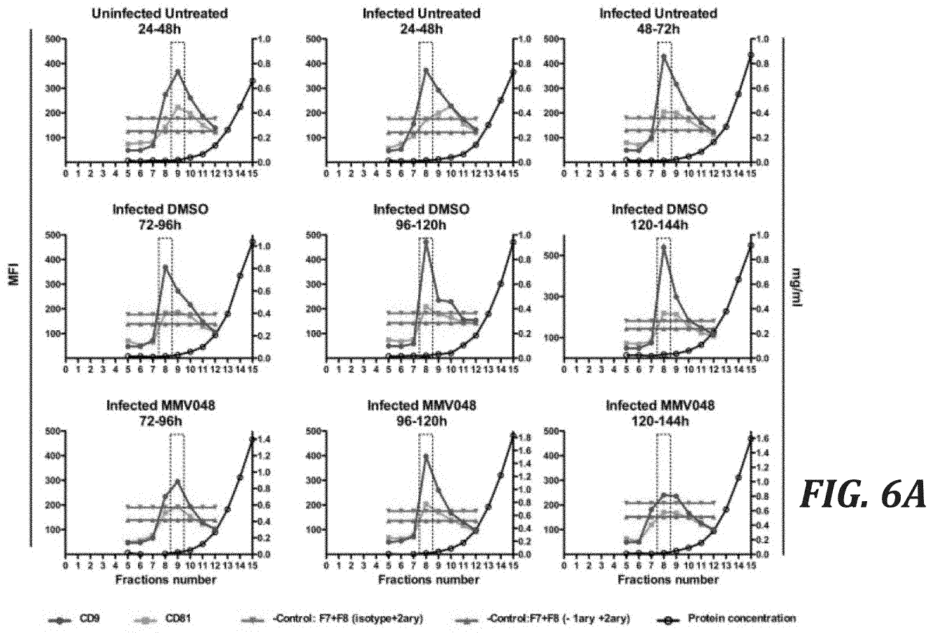

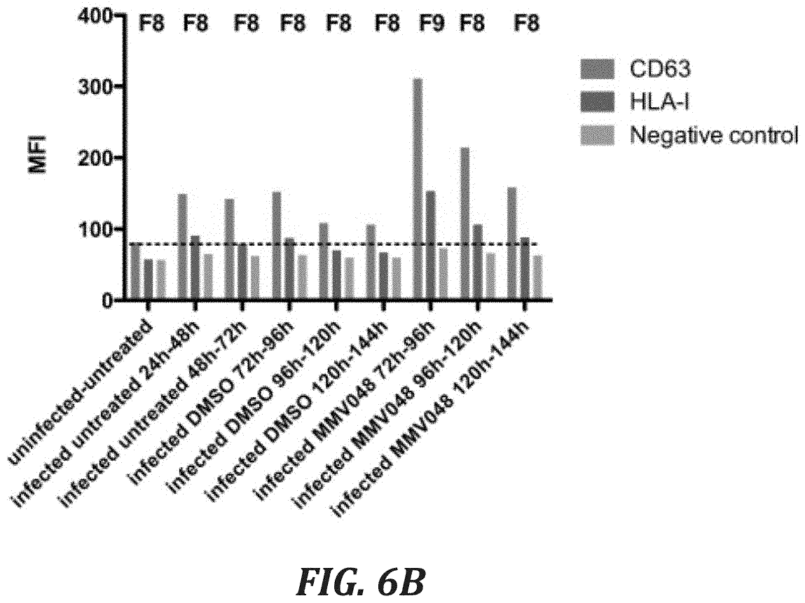

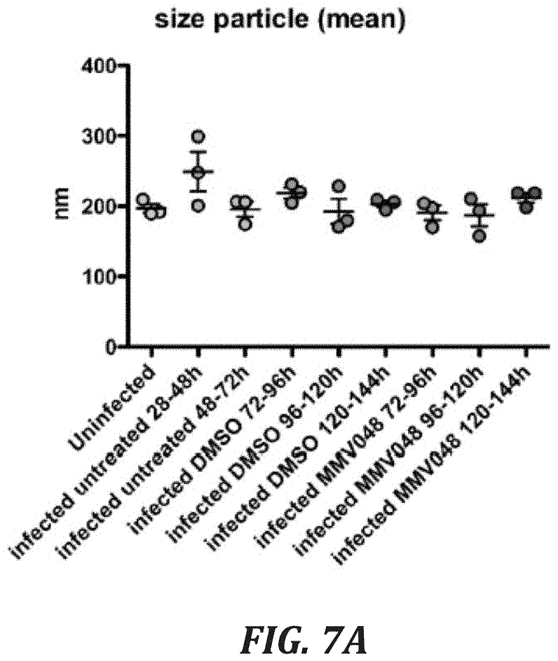

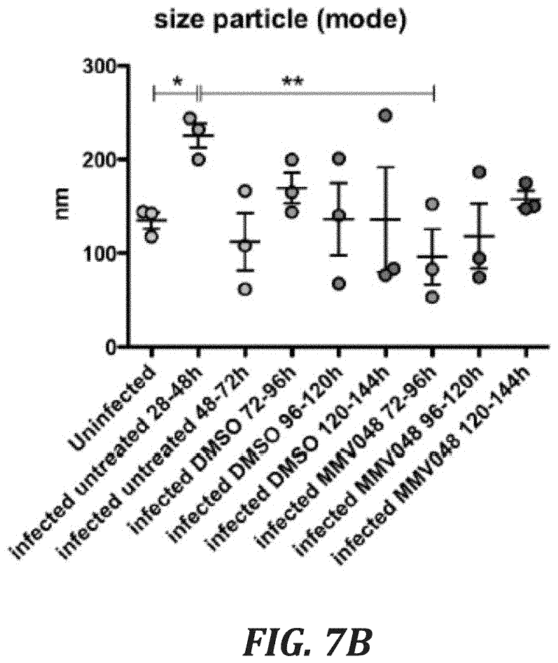

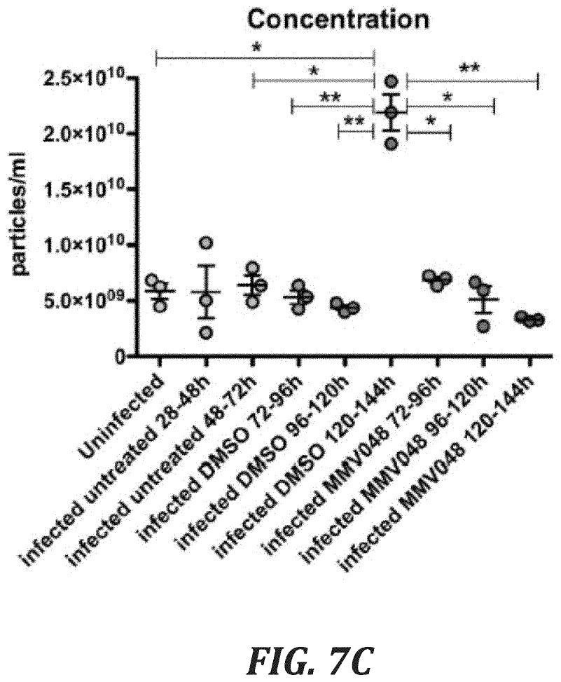

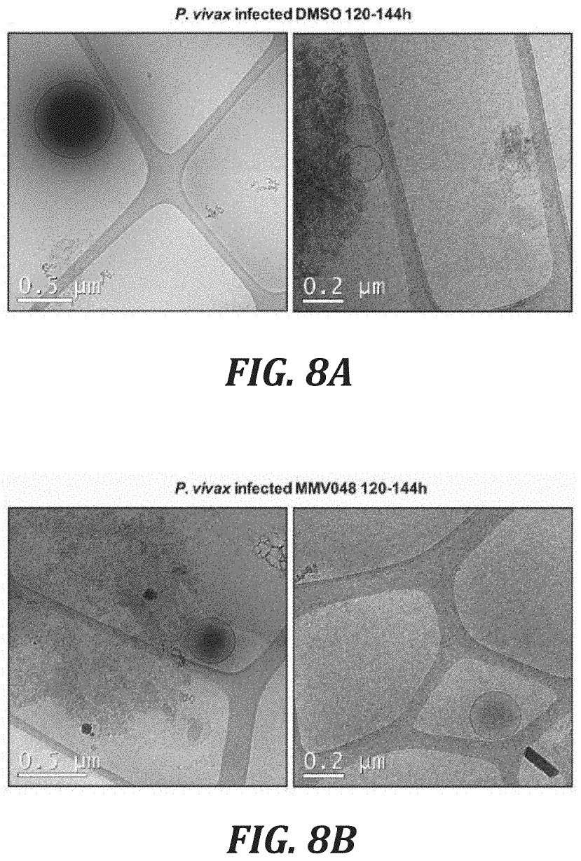

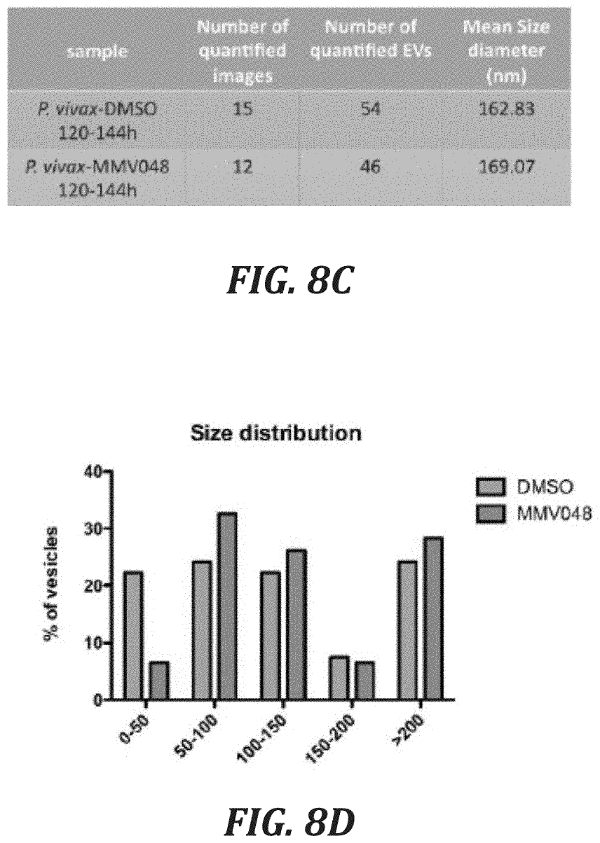

Conditioned media from in vitro cultures was used as a source for EVs isolation by SEC. Molecular characterization of SEC fractions by flow cytometry bead-based assay using the EVs markers CD9 and CD81 shows an enrichment of vesicles in fractions 7-9 (FIG. 6). Enriched EVs were clearly separated from the bulk of soluble proteins contained in the supernatant as estimated by the low protein concentration. Additional analysis of CD9⁺CD81⁺ enriched SEC fractions showed the presence of CD63 and in some cases HLA-I markers (FIG. 6B). Nanoparticle track analysis of the highest CD9/CD81/CD63 SEC fraction indicated that enriched EVs from all experimental condition has a slightly higher size than small plasma-derived EVs and exosomes (mean 187-249 nm and mode 96-169 nm) (FIG. 7).

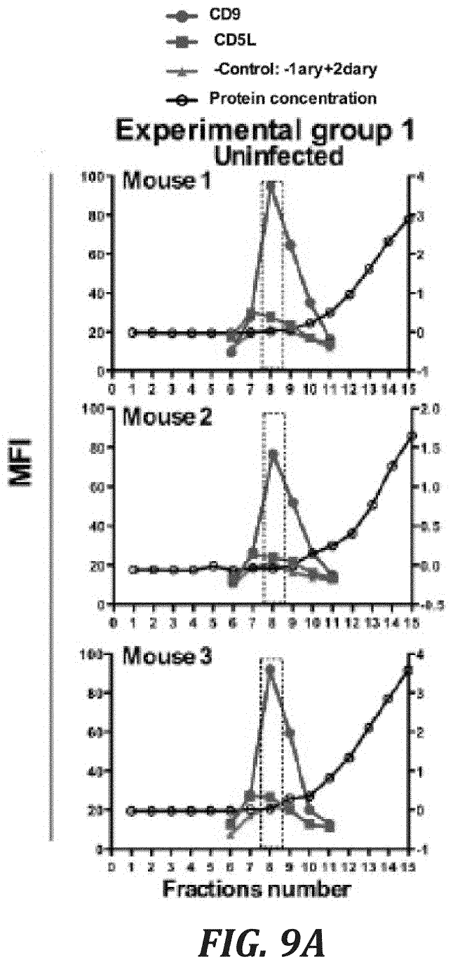

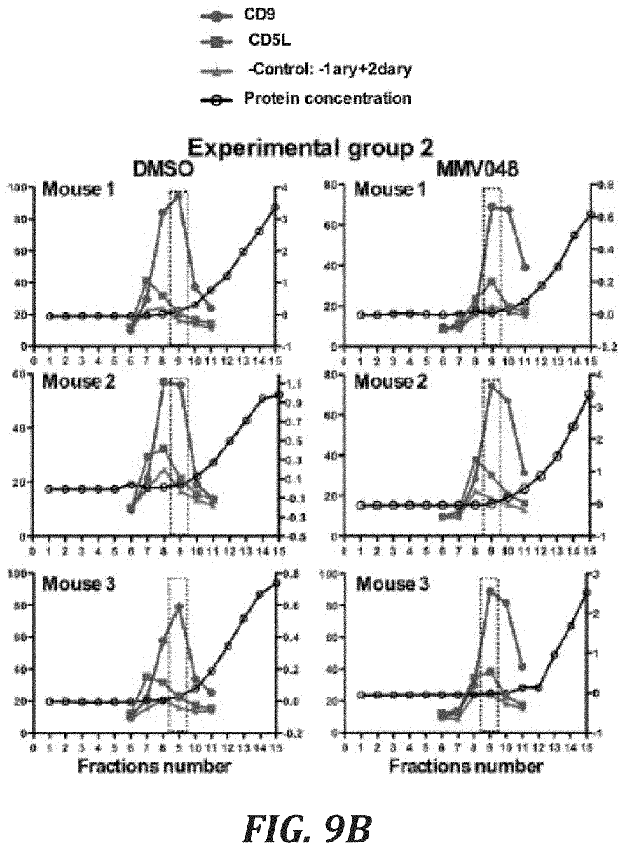

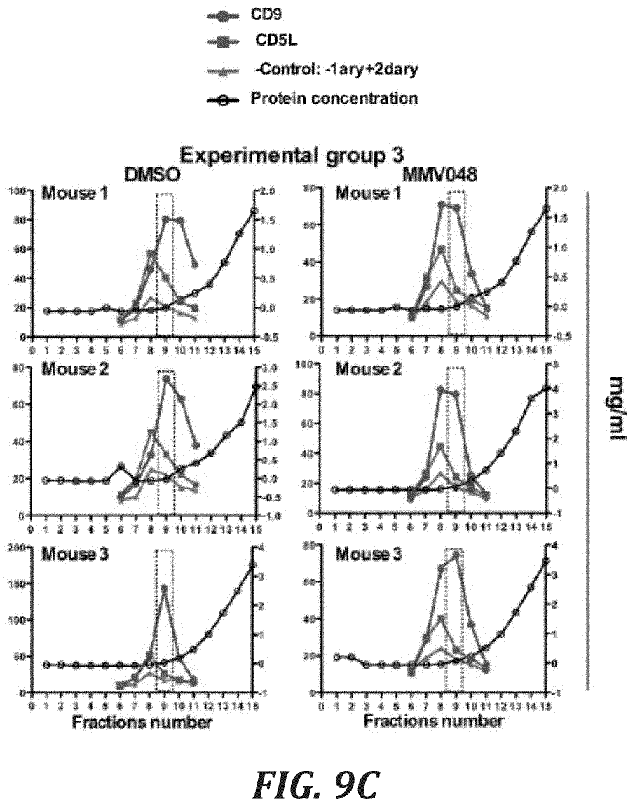

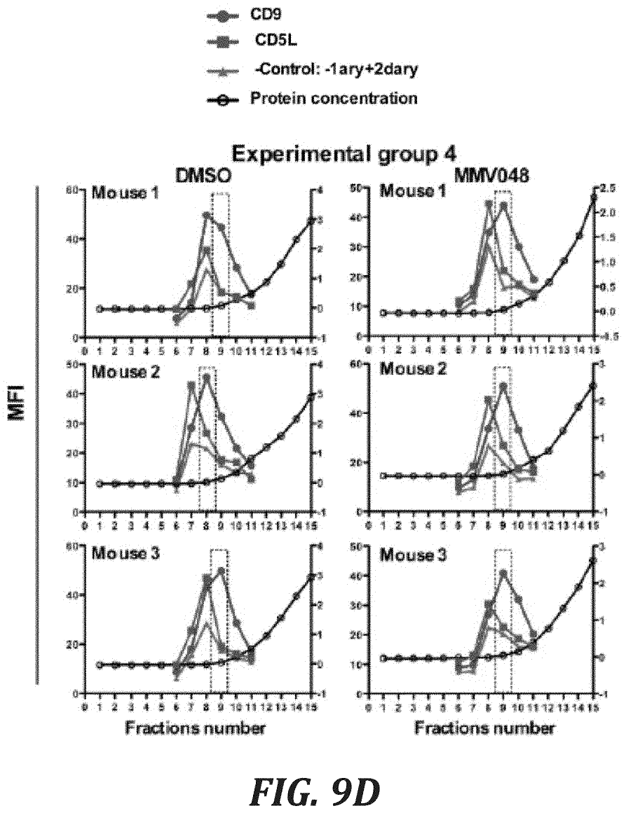

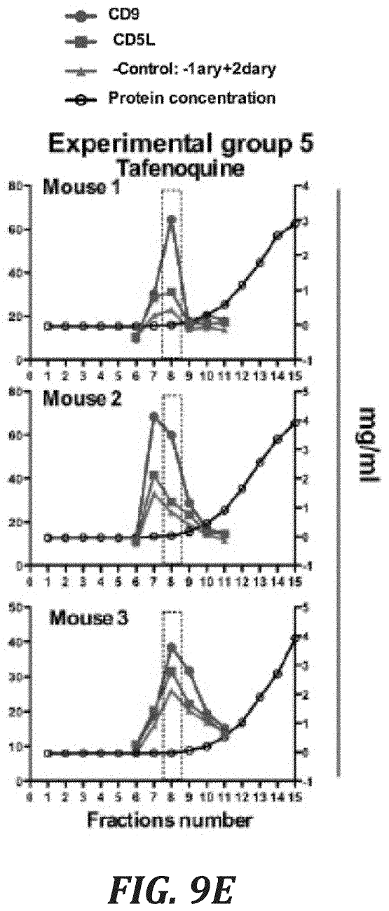

Particle concentration was homogenous among all conditions excepting EVs isolated from P. vivax infected PHH DMSO treated 120-144 h which showed a significant increased concentration. Cryo-TEM analysis of EVs from P. vivax infected PHH treated with DMSO and MMV048 at 120 h post-infection showed the presence of heterogeneous nanovesicles with a broad range of size (mean diameter of 162.8 nm and 169 nm) (FIGS. 9A-9D).

Proteomic analysis of isolated EVs showed an overall identification of 117 human proteins with varying numbers in each condition (FIG. 1C). A core of 60 proteins was found in more than 1 sample and these included major plasma soluble proteins like apolipoproteins, serum albumin, ceruloplasmin, complement factors, hemoglobins, among others. Human proteins identified were compared with the Top 100 proteins most commonly identified in EVs according to Vesiclepedia (Kalra H, et al. Vesiclepedia: A compendium for extracellular vesicles with continuous community annotation. PLoS biology 10, e1001450 (2012)). This comparison identified 8 proteins including cytosolic proteins such as myosin-9, L-lactate dehydrogenase A chain, glyceraldehyde-3-phosphate dehydrogenase, and proteins from endocytic pathways, such as ras-related Rap-lb-like protein, and membrane proteins such as sodium/potassium-transporting ATPase subunit alpha-1 and isoform 2 of annexin A2. In addition, the total human proteins identified in PHH were compared, disregarding the infection status, with three previously published proteomes from exosomes derived from in vitro cultured hepatocytes (FIG. 1D) in order to identify potential hepatocyte-specific markers associated to EVs. The comparison showed that 22, 58 and 28 proteins were common to the proteome of primary rat hepatocytes (PRH), HepAD38 Hepatitis B Virus infected and Huh-7 Hepatitis B Virus infected, respectively. A core of 12 proteins were common in all proteomes (FIG. 1D).

Seven parasite proteins in EVs were identified from P. vivax-infected PHH culture supernatants with larger numbers present in EVs collected from DMSO and MMV048-treated cultures at 120-144 hours (FIG. 1C). Interestingly, Histone H2A (PVP01_0819300) was observed in both conditions and had been previously identified in plasma-derived EVs of P. vivax infected FRG huHep mice. An Hsp70-like protein (PVP01_0515400) was identified in EVs from P. vivax infected PHH treated with MMV048 collected at 120 h-144 h (FIG. 1C). Based on the high sequence similarity, it is likely that this protein corresponds to the heat shock protein 70, putative (PVX_099315) also identified in the P. vivax infected FRG huHep mice. Of interest, a 30 kDa well conserved uncharacterized protein (PVP01_0923300) was identified which bears a signal peptide and two predicted transmembrane domains, indicating its potential association with vesicle membranes. Importantly, this protein was only detected in the EVs coming from P. vivax hypnozoites enriched cultures (treated with MMV048 at 120-144 hours post-infection) (FIG. 1C). Moreover, PVP01_0923300 was found to be slightly upregulated in a transcriptional analysis of in vitro cultured P. vivax hypnozoites when compared to schizontes/hypnozoites mixed cultures.

Identification of P. vivax Hypnozoites Biomarkers Associated to Plasma-Derived EVs in the FRG huHep Mice In Vivo Model

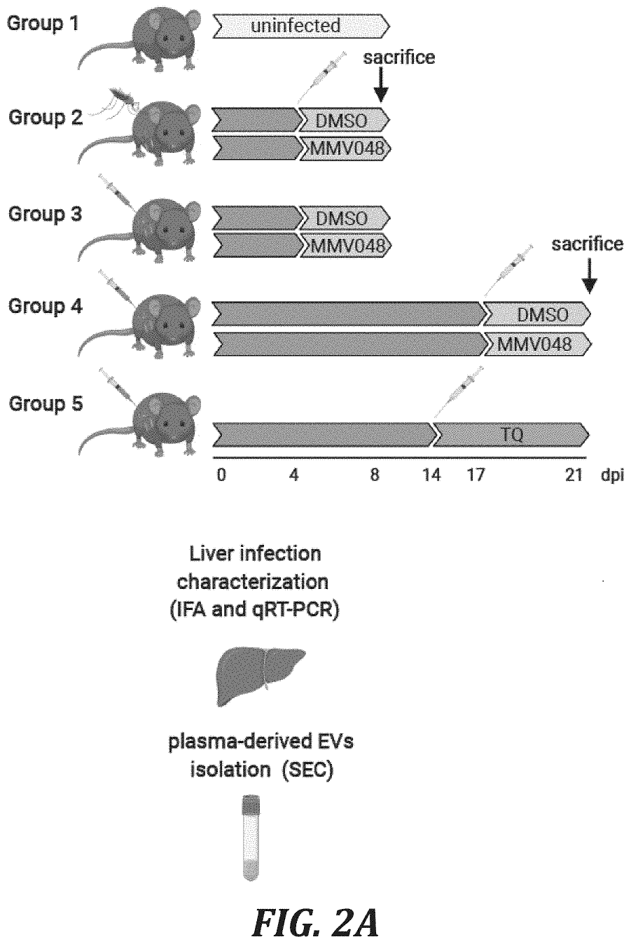

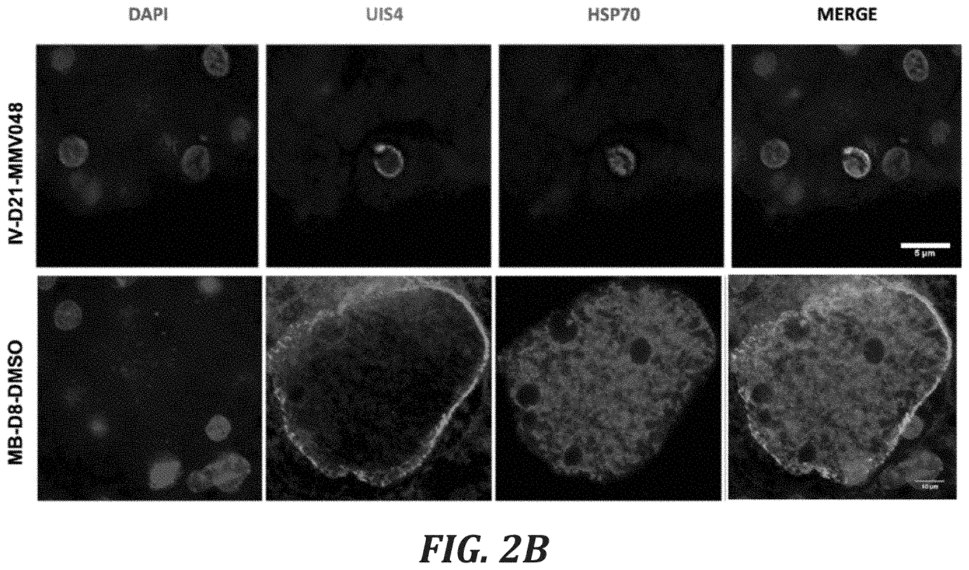

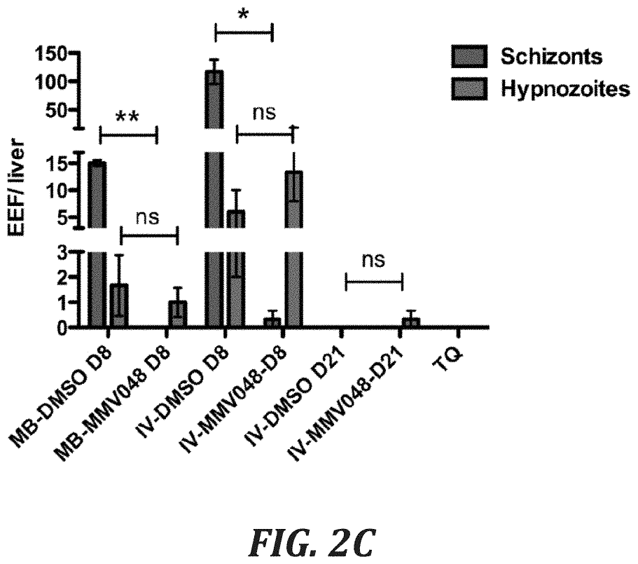

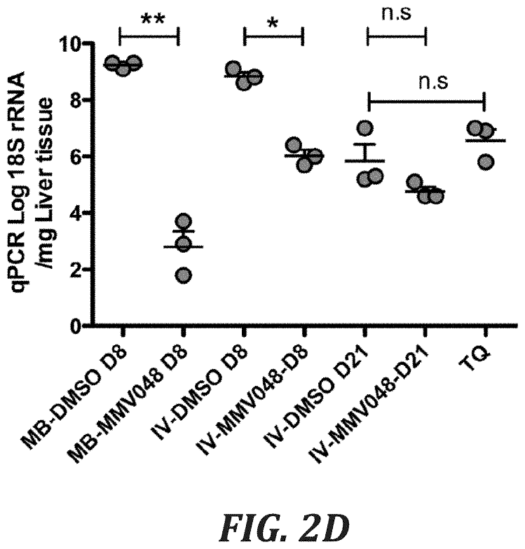

Five different groups of FRG HuHep mice were used in these studies: uninfected control animals (Group 1), mice infected with P. vivax sporozoites by mosquito bite (Group 2) or intravenous injection and further treated with MMV048 (Groups 3, 4) or with the radical cure drug Tafenoquine (Group 5) (FIG. 2A). Considering the previously established infection kinetics of P. vivax in the FRG huHEP mice model, mature hypnozoites were detected at 8 days post-infection. In addition, hypnozoites can reactivate and generate a second wave of schizontes at 21 days post-infection. Taking these observations into account, end-point analysis was performed at 8 days post-infection for both, group 2 and 3. In addition, an analysis at 21 days post-infection in mice infected by intravenous injection (Group 4) was included reasoning that treatment with MMV048 from day 17-21 days post-infection would remove the second generation of schizonts, giving an additional window of time for resistant and growing hypnozoites to secrete EVs in circulation. Liver sections were evaluated by IFA for the presence of the parasitophorous vacuole membrane protein UIS4 and the cytosolic HSP70. Results clearly showed small liver stage hypnozoites in the liver of MMV048-treated mice and large schizonts in DMSO-treated controls (FIG. 2B). Quantification of the number of schizonts and hypnozoites showed that MMV048-treated mice from groups 2, 3 and 4 contained a significantly lower number of schizonts and a similar number of hypnozoites when compared to respective untreated control mice (FIG. 2C). Notably, livers from intravenously infected mice analyzed at day 8 showed a larger number of liver-stage forms when compared to mice from mosquito bite infections. Livers collected after 21 days post-intravenous infection showed no schizonts in both MMV048- and DMSO-treated mice and only one hypnozoite was detected in MMV048-treated mice. These results indicate that hypnozoites failed reactivation after 8 days and a second generation of schizonts did not form. Quantification by RT-qPCR analysis of 18S rRNA in liver tissue showed a significantly reduced parasite load in MMV048-treated mice when compared to DMSO-treated controls in both mosquito bite infected and intravenous infection analyzed 8 days post-infection (FIG. 2D), supporting the results observed by IFA. In spite of the absence of livers stages in IFA at day 21, parasite load was found similar to levels of hypnozoites-enriched livers from group 3 with no differences in MMV48- and DMSO-treated animals. As expected for radical cure treatment, no liver forms were quantified in the liver of tafenoquine-treated animals, although RT-qPCR data indicated presence of parasite 18S rRNA in this condition.

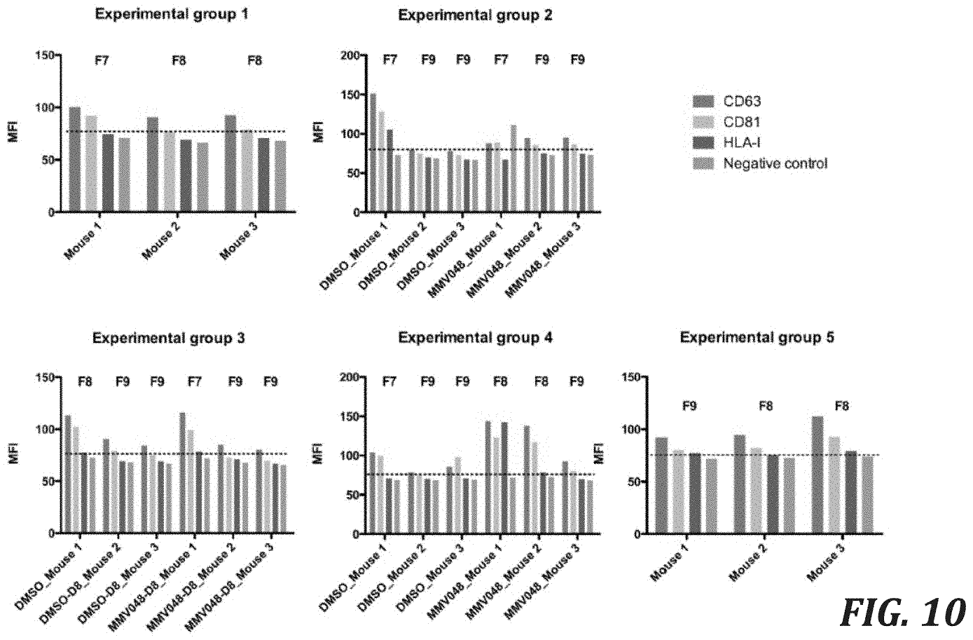

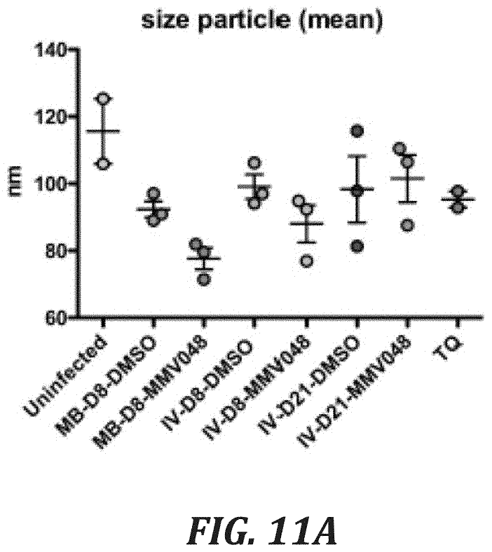

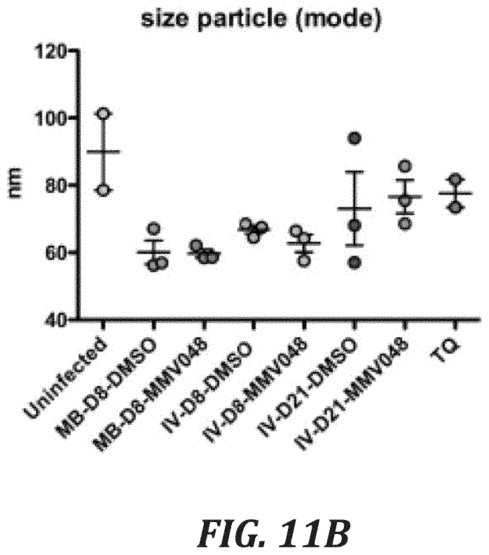

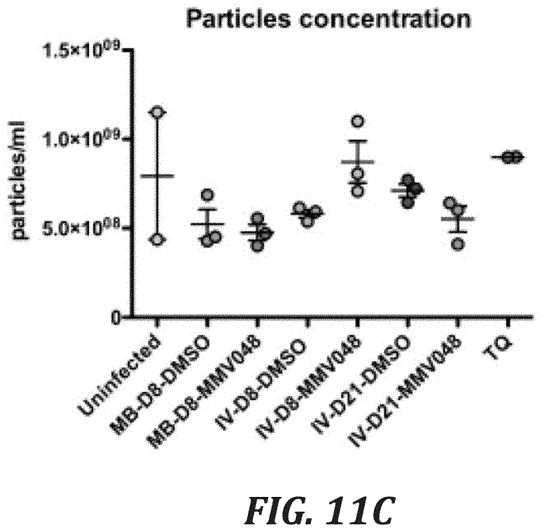

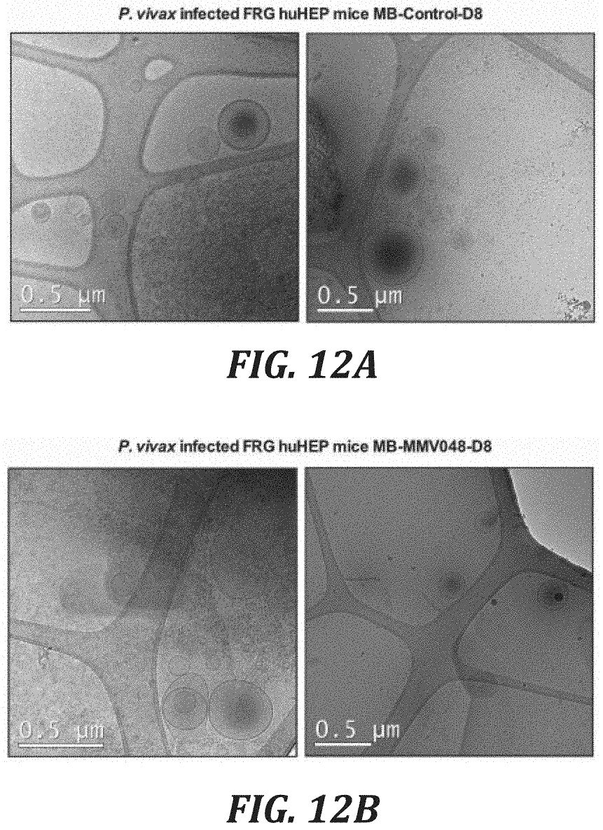

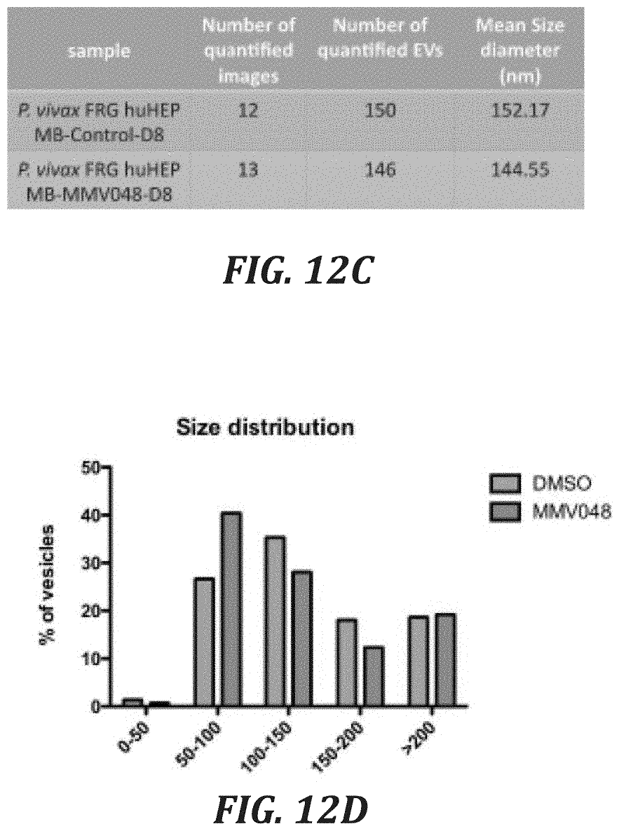

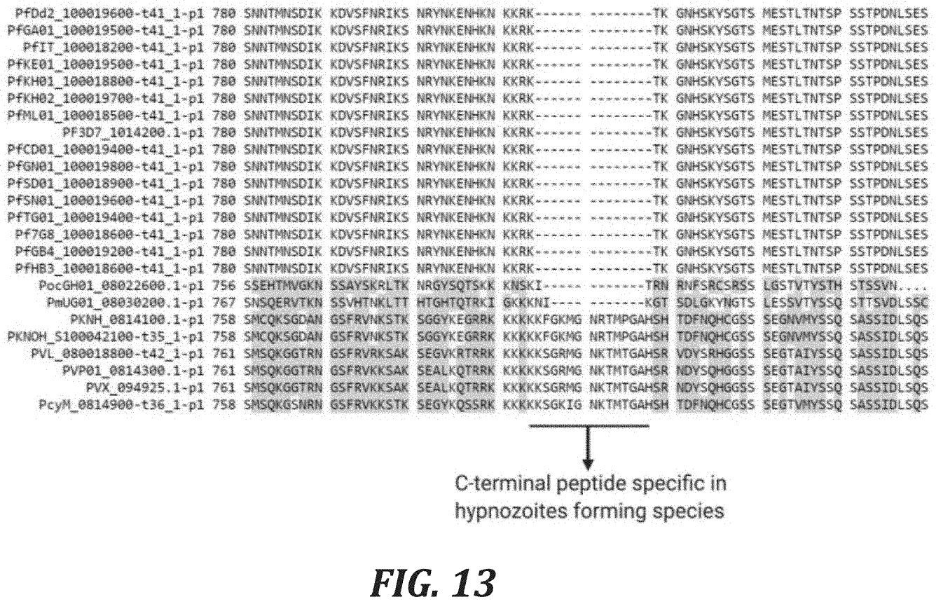

Plasma from P. vivax infected FRG huHep mice from all experimental groups was used as a source of circulating EVs, which were further isolated by SEC. Molecular characterization of SEC fractions by flow cytometry bead-based assay showed an enrichment of CD9+ and CD5L vesicles between Fraction 7, 8, and 9, a profile that is in agreement with small vesicles or exosomes elution profiles reported by qEV columns manufacturer (FIGS. 10A-10E). Importantly, enriched vesicles were clearly separated from the bulk of plasma soluble proteins as estimated from the protein concentration. Additional analysis of CD9⁺ CD5L⁺ enriched SEC fractions showed the presence of CD63 and CD81 (FIGS. 11A-11C). Particles size distribution and concentration of the highest CD5L/CD9/CD63 SEC fractions estimated by NTA showed that enriched particles had a size between 55-100 nm (mode), 75-120 (mean) and a concentration in the range of 5×10⁸ and 1.2×10⁹ particles/ml (FIG. 12). Particle size correspond to that one of small vesicles which includes exosomes and plasma membrane-derived smalls EVs. Complementary analysis by Cryo-TEM of EVs enriched SEC fractions from experimental group 2 mice (MB-D8) showed the presence of double membrane electron dense nanovesicles of homogenous size distribution (mean size: DMSO-treated 152.17 nm and MMV048-treated 144.5 nm)) (FIG. 13).

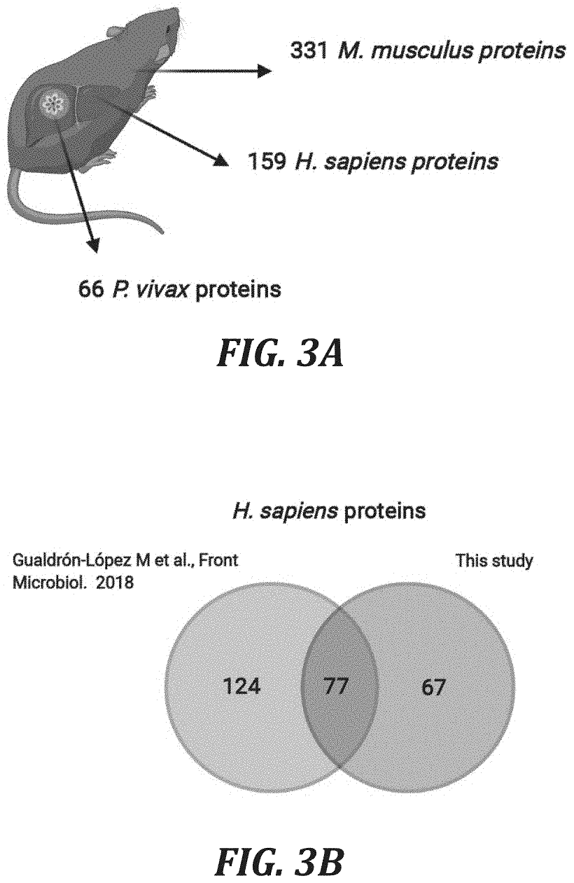

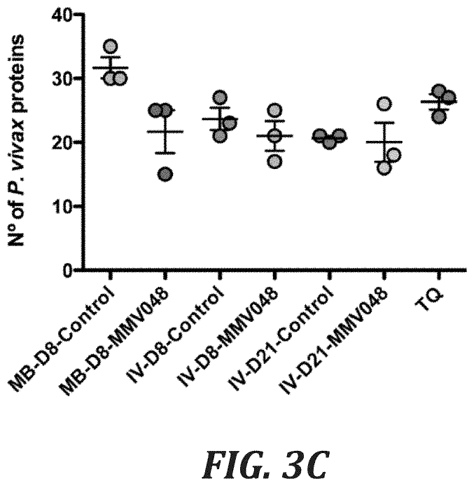

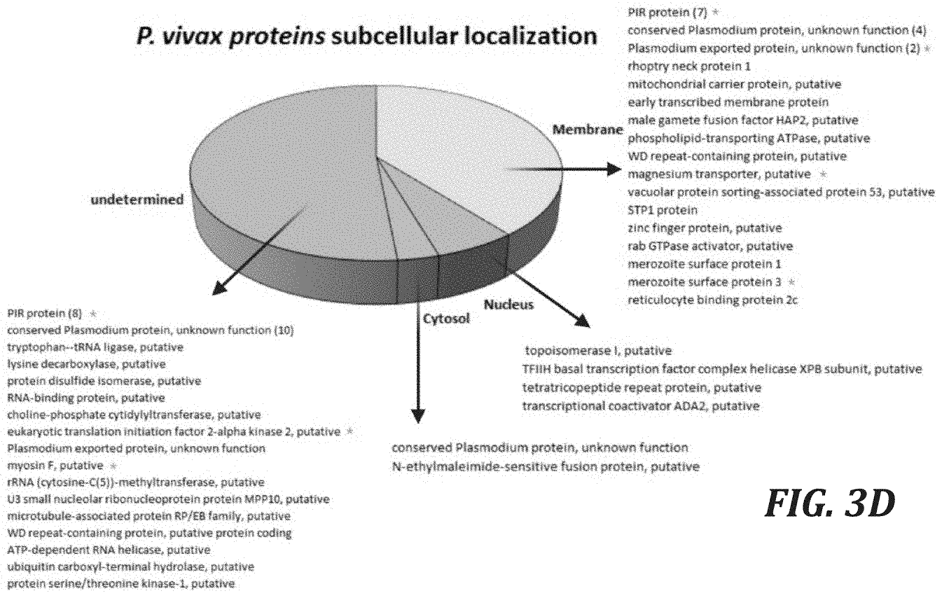

Proteomic characterization of plasma-derived EVs from all groups of P. vivax infected FRG huHep mice showed that EVs-enriched SEC fractions contained proteins from the three species: 159 human, 331 mouse and 66 P. vivax proteins (FIG. 3A). Human proteins include 18 proteins from the top 100 EVs most abundant proteins as reported by Vesiclepedia. From the whole human proteome obtained 20, 63, and 23 proteins were previously reported in proteomes from human hepatocytes, respectively, and 77 in P. vivax infected FRG huHep mice. EV markers from mouse origin included CD9, CD5L, syntenin-1, integrin a and b and Na/K ATPase and from human origin annexin A2, 14-3-3 protein epsilon, periredin-1, Rap-lb and Rab-10, among others.

The number of P. vivax proteins identified were similarly distributed in all experimental groups, with a tendency of smaller number of proteins in EVs from MMV048-treated mice of MB-D8 (Group 2) and IV-D8 (Group 3) when compared to DMSO-treated controls, although not statistically significant different (FIG. 3C). Subcellular localization prediction indicates that 39% of the total proteins identified were associated to membranes, 3% to cytosol, and 6% to nucleus (FIG. 3D). The remaining 52% proteins were not assigned to a specific cell compartment in our analysis. These proteins include 8 members of the VIR family, 10 conserved Plasmodium proteins of unknown function and several proteins with a large variety of predicted functions. Membrane proteins includes 7 VIR family members, an early transcribed protein (ETRAMP), vacuolar sorting-associated protein 53, Rab GTPase activator, reticulocyte binding protein 2c, merozoite surface protein 1 and 3, among others. Cytosolic proteins include a conserved Plasmodium protein of unknown function and N-ethylmaleimide-sensitive fusion protein involved in protein transport. Nuclear proteins include proteins associated to transcription like topoisomerase I, TFIIH basal transcription factor complex helicase XPB subunit, a tetratricopeptide repeat protein and transcriptional coactivator ADA2 (FIG. 3D). Interestingly, 20 of the P. vivax proteins identified were found to be specific of the hypnozoite-forming species (FIG. 3D).

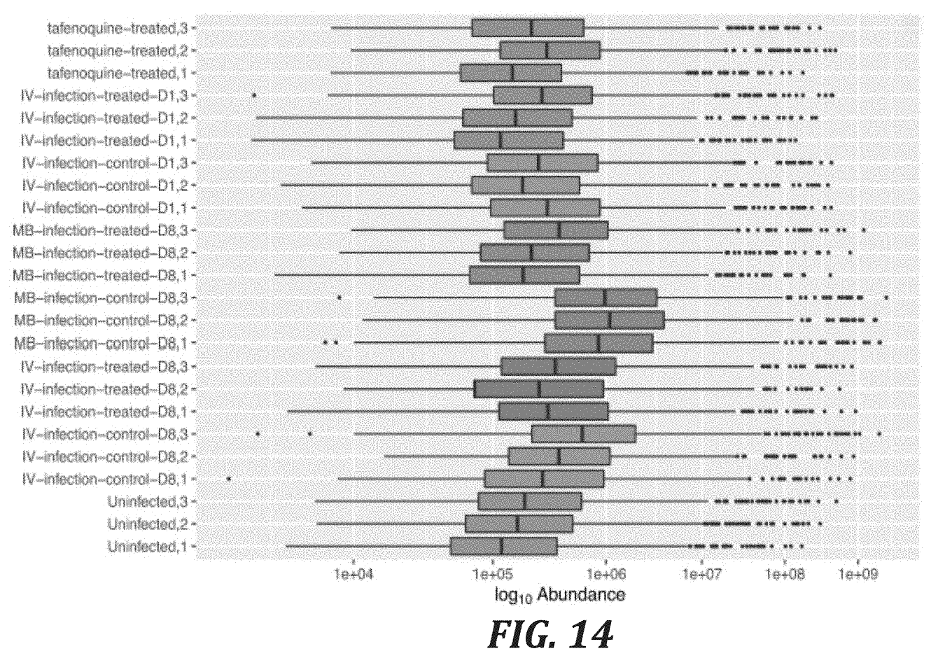

Screening of hypnozoites-specific proteins associated to EVs was done by first excluding proteins identified in the experimental group treated with Tafenoquine and then comparing PR and AEpS proteomic data between MMV048-treated infected mice and DMSO-treated control-infected mice. This analysis showed the presence of six potential candidate proteins. These were detected in MMV048-treated mice which plasma sample was collected after 8 days (mosquito bite and IV infection) and 21 days post-infection and absent in its respective intragroup DMSO-treated controls, as well as, in samples from intergroup DMSO-treated mice (Table 1). Importantly, one protein (PVP01_0814300) was found in three biological replicates of the treated mice from group 2 (MB-MMV048-8D) and in one mouse of the treated group 4 (IV-MMV048-21D). A summary of the six protein candidates indicating orthology, and gene expression in the different P. vivax developmental stages, is described in Table 1. Among the hypnozoites biomarker candidates the most robust candidate protein (PVP010814300) corresponds to HAP2, a well-conserved Plasmodium protein previously found to be involved in membrane fusion during fertilization and important for parasite transmission. Considering the high sequence similarity of PVP01_0814300 with its orthologs in other human malaria parasites, the protein sequence of all Plasmodium orthologous were analyzed in order to identify possible protein segments exclusively present in P. vivax with potential to be exploited as a target for the detection of EVs derived from P. vivax hypnozoites, without detecting the confounding prudence of other malaria parasite species. Results showed that beside the high sequence similarity, a C-terminal peptide was found exclusively in P. vivax and in hypnozoites-forming species P. cynomolgy and P. knowlesi (FIG. 14).

### Discussion

Here, the proteomes were explored of extracellular vesicles (EVs)-derived from MMV048-treated primary human hepatocyte (PHH) in vitro cultures and plasma from FRG huHep mice infected with P. vivax sporozoites in which there was an enrichment of P. vivax hypnozoites forms showing no schizont forms. Results identified parasite proteins exclusively associated with hypnozoite stages, thus indicating that this approach should accelerate the development of tools to detect asymptomatic patients harboring hypnozoites in their liver, a major obstacle in malaria elimination.

MMV048 has been proved to have potent activity against asexual, transmission and liver stages of Plasmodium spp. in preclinical studies. The target of this drug is the PI4K, a protein involved in membrane recruitment and dynamics during asexual replication. Interestingly, when applied in radical cure mode this drug has been proved ineffective against liver hypnozoites in P. cynomolgi, both in vitro and in vivo, likely due to the low expression of the PI4K drug target in this parasite stage, as has been previously suggested for P. vivax hypnozoites. Thus, MMV048 should eliminate all replicating P. vivax liver stages while leaving hypnozoites unaffected a result that was observed both in vitro and in vivo (FIGS. 1A, 1B and FIGS. 2B, 2C and 2D), thus validating this approach for searching hypnozoite biomarkers.

Proteomics analysis of EVs from PPH in vitro cultures enriched in P. vivax hypnozoites identified six parasite proteins and this low number is in agreement with the low amount of total human proteins identified. Despite this limitation, parasite proteins previously identified were found in the proteome of P. vivax FRG huHEP mice (HSP70 and Histone H2A). Of interest, identified was a highly syntenic and conserved parasite protein of unknown function, PVX_091752=PVP01_0923300, a membrane protein secreted in EVs from P. vivax hypnozoite-infected PHH and whose transcripts were found to be unregulated in hypnozoites vs. schizonts. Its value as a biomarker specific for P. vivax hypnozoites, however, will require further investigations.

In contrast to the results obtained from PHH in vitro cultures, overall proteomic analysis of plasma-derived EVs in the FRG huHEP mice model identified 66 parasite proteins. These included proteins involved in lipids and ions transport, such as a putative Mitochondrial Carrier protein and Phospholipid-transporting ATPase as well as a putative magnesium transporter. In addition, detected were proteins participating in membrane trafficking like the vacuolar sorting-associated proteins p53 putative and rab GTPase activator (FIG. 3D). Metabolic enzymes, DNA remodeling and RNA binding proteins, were also present. This agreed with a functional association with EVs as these nanovesicles are characterized by the presence of multiple membrane, cytosolic and nuclear proteins. Of interest, a large component of the parasite proteome was represented by several members of variant vir gene superfamily (FIG. 3D). The association of VIR proteins with EVs could be due to its sorting in host-derived EVs or could occur as structural components of parasite-derived EVs. Of note, several proteins identified in plasma-derived EVs from infected FRG huHep mice are also found in merozoites stages (MSP1, MSP3, RBP2c, Roptry neck protein) (FIG. 3D). Such association with EVs could indicate that hepatocytes infected with mature exoerythrocytic schizonts secrete EVs that reach the circulation.

To identify hypnozoite-specific proteins associated to EVs we mined the proteome data from PPH in vitro cultures enriched in P. vivax hypnozoites and from MMV048-treated mice and performed intra and inter experimental group comparisons. In addition, parasite proteins were excluded that were detected in the Tafenoquine-treated mice to exclude false positive proteins. Analysis of both, PR and AEpS proteomic data revealed one interesting candidate protein, PVP01_0814300 (gamete membrane fusion protein, HAP2) fulfilling the above-mentioned criteria (Table 1). HAP2 is a well conserved protein with structural similarity to the class II viral fusion proteins involved in fusogenic membrane process in a wide range of organism including plants, non-pathogenic and pathogenic protists, comprising Plasmodium spp. Despite the low Mascot score, the PR observed in the mosquito bite infected group for HAP2 protein was found statistically significant. Hypnozoite expression of HAP2 in P. vivax is further supported by a previous transcriptional analysis that has shown that this gene is expressed in hypnozoite-enriched, infected hepatocyte cultures. The presence of a transmembrane domain and its molecular function in membranes fusion events indicates that its association with EVs is plausible and could play biological functions in the parasite EVs biogenesis process. The fact that HAP2 is also expressed in male gametes and is well conserved in other Plasmodium spp, could compromise its value as a biomarker specific for P. vivax hypnozoites. However, that cannot only be excluded during hypnozoite stage infection of hepatocytes this protein gets exported and associated with EVs in a stage-specific manner. This will require further investigation. Importantly, a C-terminal peptide was found exclusively present in hypnozoite-forming Plasmodium species, indicating that it might be possible to develop species-specific reagents to detect HAP2 only from relapsing malaria species.

Analysis performed in the AEpS data identified other putative hypnozoite-specific protein candidates. Although these proteins were found only in one infected FRG huHep mouse at day 8 post-infection, this animal showed the largest number of hypnozoites, supporting a possible association with EVs-derived from this developmental stage. Of these proteins, only PVP01_0930100 (proliferating-cell nucleolar antigen p120, putative) was identified in a previous P. vivax hypnozoite transcriptome. However, it has also been found expressed in P. vivax intraeritrocytic stages indicating that it is not specific to hypnozoites. In addition, this protein has a predicted rRNA (cytosine-C(5))-methyltransferas enzymatic activity and lacks predicted transmembrane domains, indicating that it association to EVs is in the lumen (Table 1).

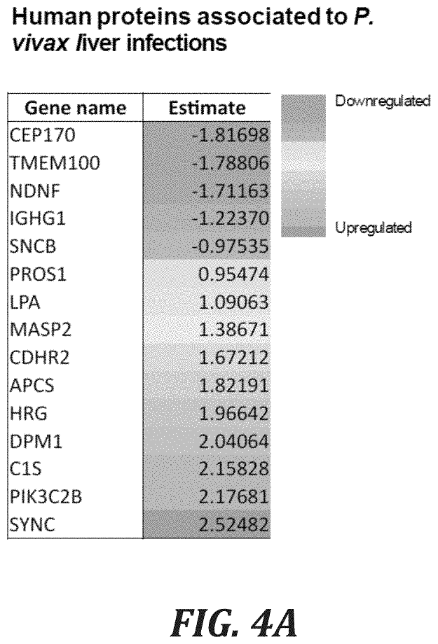

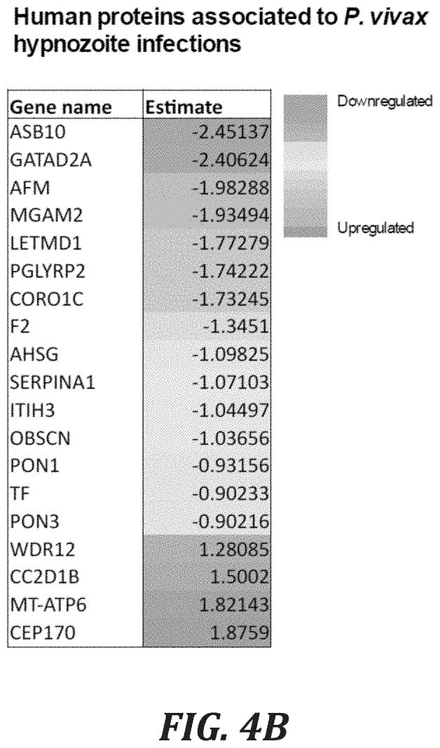

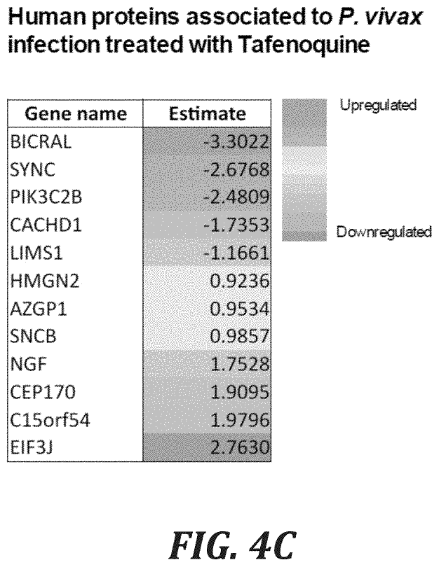

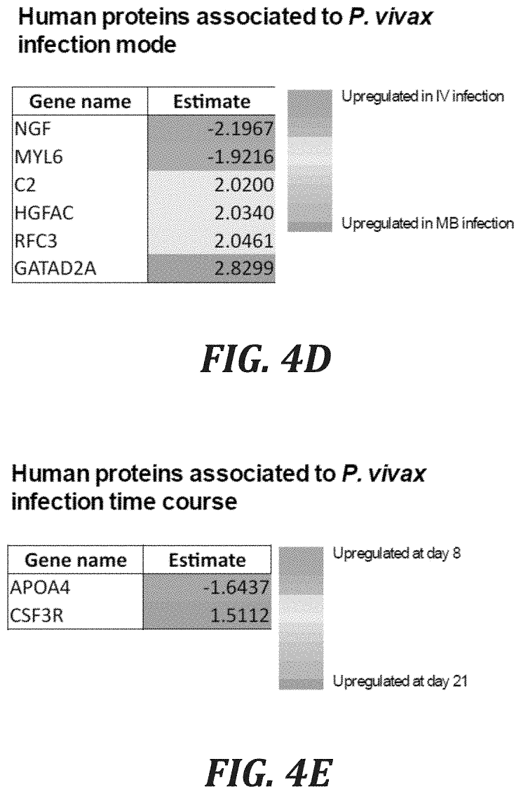

The potential of EVs molecular contents as biomarkers of disease is attributed to the fact that they are a fingerprint of the cell of origin. In this sense, the assessment of statistical differences between the human components of plasma-derived EVs in the FRG huHep mice throughout the different experimental condition used in this study, aimed to gain additional knowledge of the physiological status of human hepatocytes during this infection. These results reflect that infected hepatocytes respond to P. vivax infection secreting EVs with signatures of inflammation (FIG. 4A) being hypnozoite infections immunologically more silent that replicating schizonts, as expected (FIG. 4B). Noteworthy, a member of the PI3K family (PIK3C2B) was found upregulated in EVs from Tafenoquine-treated mice when compared to infected and DMSO treated mice (FIG. 4C). This could imply that Tafenoquine can induce increased expression and secretion in EVs of PIK3C2B provoking alterations in hepatocytes cell-signaling pathways involved in proliferation, cell survival and intracellular protein trafficking during Tafenoquine metabolism in the liver. The increased association of this protein with EVs upon Tafenoquine treatment is interesting and will need further investigation.

In summary, the results herein show that LC-MS/MS-based proteomics of EVs generated both, in in vitro and in vivo models of P. vivax upon treatment with the schizonticidal experimental drug MMV048, identified parasite proteins with the potential of being released within EVs in a hypnozoite stage-specific manner. Thus, this study set the stage for the discovery of specific biomarkers of asymptomatic P. vivax liver infections and should advance the development of diagnostic tools for the identification of asymptomatic hypnozoite carriers in human populations.

### Methods

P. vivax Infection of Primary Human Hepatocytes (PHH)

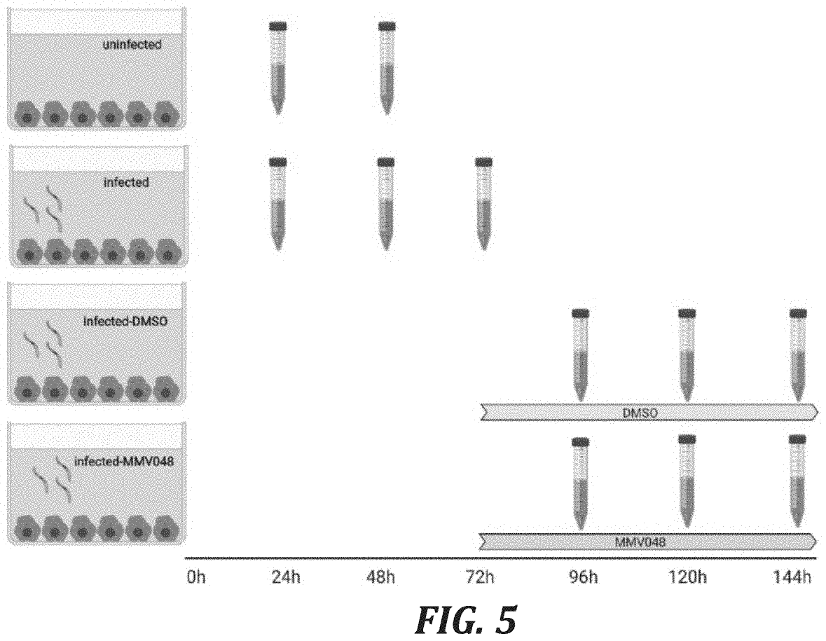

Primary human hepatocytes (BioIVT) were plated and infected in chamber slides as previously described (Schafer C, et al. A Humanized Mouse Model for Plasmodium vivax to Test Interventions that Block Liver Stage to Blood Stage Transition and Blood Stage Infection. Science 23, 101381 (2020)). Briefly, 8-well Permanox™ slides (LabTek) were coated with collagen (Advanced Biomatrix) and seeded with 1.25×10⁵ viable cells using InVitroGRO™ CP medium (BioIVT) supplemented with 2% FBS without antibiotics (plating media). Four hours later, plating media was replaced with InVitroGRO™ CP containing antibiotics (1× Torpedo Antibiotic Mix with 10 μM gentamycin) without serum (maintenance media). Two days after plating, cells were infected with freshly dissected P. vivax salivary gland sporozoites (MOI=1.0) for four hours in maintenance media supplemented with 20% FBS. Cells were washed once, and maintenance media replaced daily thereafter and stored at −80° C. for EV analysis. After day 3 post-infection, cultures were treated with 0.1 μM MMV048 or 0.1% DMSO as vehicle (FIG. 5). On day 6-post infection, cells were fixed and analyzed by IFA. Total extraerythrocytic forms (EEF) and schizont/hypnozoite numbers and ratio were quantified.

P. vivax Infection of FRG huHep Mice

All animal procedures were conducted in accordance with and approved by the Center for Infectious Disease Research Institutional Animal Care and Use Committee (IACUC). The Center for Infectious Disease Research IACUC adheres to the NIH Office of Laboratory Animal Welfare standards (OLAW welfare assurance #A3640-01). Female FRG KO mice engrafted with human hepatocytes (FRG KO huHep) were purchased from Yecuris Corporation (Oregon, USA). P. vivax infections of FRG huHEP were performed as previously described. Briefly, mice were divided in five experimental groups. Group 1 (3 mice) was not infected. Group 2 (6 mice) was inoculated with P. vivax sporozoites by the bite of 20 mosquitos and euthanized after 8 days post-infection (dpi). Group 3 (6 mice) was infected by intravenous injection of P. vivax sporozoites (0.8 million) and euthanized after 8 dpi. Group 4 (6 mice) was infected by intravenous injection of P. vivax sporozoites (1 million sporozoites) and euthanized after 21 dpi. Group 5 (3 mice) was infected by intravenous injection (1 million sporozoites) and treated with Tafenoquine (10 mg/kg) at 14 dpi and euthanized at 21 dpi. MMV048 (Novartis) mice treatment (30 mg/kg) was performed as follows: three mice from Groups 2 and 3 received intravenous injections of the drug at 4 dpi; three mice from Group 4 received intravenous injections of the drug at 17 dpi. Three DMSO-treated animals were used as controls in groups 2, 3 and 4. After euthanasia, livers were extracted for characterization by IFA and RT-qPCR and whole blood was extracted by cardiac venipuncture for plasma collection as previously described (Schafer C, et al. A recombinant antibody against Plasmodium vivax UIS4 for distinguishing replicating from dormant liver stages. Malar J 17, 370 (2018)).

### Immunofluorescence Analysis

IFA analysis of PHH and liver sections of FRG huHep mice was performed as described (Schafer C, et al. A Humanized Mouse Model for Plasmodium vivax to Test Interventions that Block Liver Stage to Blood Stage Transition and Blood Stage Infection. iScience 23, 101381 (2020); Schafer C, et al. A recombinant antibody against Plasmodium vivax UIS4 for distinguishing replicating from dormant liver stages. Malar J 17, 370 (2018)). Briefly, culture cells and liver tissue were fixed in 4% paraformaldehyde in TBS for 10 min (PHH) and 16 hours (liver sections). PHH were blocked and permeabilized in TBS with 2% bovine serum albumin and 0.2% Triton-X-100 (PBS/BSA/Triton). FRG huHep fixed livers were sectioned in 50 μm thick sections in a vibratome. Tissue sections were then permeabilized in 0.25% Triton X-100 and 3% H₂O₂ for 30 min followed by a blocking step in 5% skim milk in TBS for 1 hour at RT. Both, PHH and mouse liver double staining were performed using rabbit anti-P. vivax HSP70 primary antibodies and a mouse monoclonal anti-P. vivax UIS4 antibody. Fluorescent staining was achieved by incubation with Alexa Fluor-conjugated secondary antibodies (Thermo Fisher) specific to rabbit (Alexa Fluor™ 594) and mouse (Alexa Fluor™ 488) IgG for 2 hours at RT. After one wash in TBS, nuclei were stained with DAPI for 10 minutes at RT and samples mounted with ProLong anti-fade Mountant (Thermo Fisher). Images were acquired using Olympus 1x70 DeltaVision deconvolution microscopy.

18S qRT-PCR Analysis of Parasite Load

Liver parasite load from infected FRG huHEP mice was quantified as previously described (Flannery E L, et al. Assessing drug efficacy against Plasmodium falciparum liver stages in vivo. JCI Insight 3, (2018)).

### Isolation of Extracellular Vesicles (EVs)

Three mL of frozen culture supernatants from infected, uninfected, MMV048-treated and untreated cells were collected according to the time course shown in FIG. 5. Supernatants were thawed on ice and centrifuged at 500×g for 10 min, then at 2000×g for 10 min and further concentrated in an Amicon 10 kDa (Millipore) to 1 mL. EVs were purified by size exclusion chromatography (SEC) using commercial 10 mL Sepharose (q-EV iZON) following manufacturer instructions. SEC fractions were molecularly characterized for the presence of CD9 and CD81 EVs markers by bead-based flow cytometry (BBA) using hybridoma anti-human CD9, clone V51/20) and hybridoma anti-human CD81, clone 5A6, respectively (Thery C, Amigorena S, Raposo G, Clayton A. Isolation and characterization of exosomes from cell culture supernatants and biological fluids. Curr Protoc Cell Biol Chapter 3, Unit 3 22 (2006)). CD9⁺ CD81⁺ SEC fractions were further tested for CD63 (anti-human CD63, Immunostep 63PU-01MG) and HLA (anti-human HLA-ABC, Invitrogen 14-9983-82) by BBA. In addition, EVs-enriched SEC fractions were analyzed by nanoparticle track analysis and Cryo-Electronmicroscopy (de Menezes-Neto A, et al. Size-exclusion chromatography as a stand-alone methodology identifies novel markers in mass spectrometry analyses of plasma-derived vesicles from healthy individuals. Journal of Extracellular Vesicles 4, 27378 (2015)). Protein concentration was determined by BCA (Thermo Scientific).

Plasma-derived EVs from uninfected and P. vivax-infected, MMV048 treated or untreated mice were thawed on ice, centrifuged at 2000×g for 10 min. EVs from supernatants were purified by SEC using commercial Sepharose (q-EV iZON) following manufacturer instructions. SEC fractions were characterized by bead-based flow cytometry (BBA) for the presence of CD9 (Abcam ab92726) and CD5L (Abcam ab45408). CD9⁺CD5L⁺SEC fractions were further tested for CD63, CD81 and MHCI by BBA using antibodies set mentioned above (Thery C, Amigorena S, Raposo G, Clayton A. Isolation and characterization of exosomes from cell culture supernatants and biological fluids. Curr Protoc Cell Biol Chapter 3, Unit 3 22 (2006)). In addition, EVs enriched SEC fractions were analyzed by nanoparticle track analysis and cryo-Electronmicroscopy (de Menezes-Neto A, et al. Size-exclusion chromatography as a stand-alone methodology identifies novel markers in mass spectrometry analyses of plasma-derived vesicles from healthy individuals. Journal of Extracellular Vesicles 4, 27378 (2015)).

### Experimental Design and Statistics

### Liquid Chromatography Tandem Mass Spectrometry (LC-MS/MS)

EVs from P. vivax Infected PHH

A unique EVs sample per condition and infection time point was processed for LC/MS-MS. Conditions included uninfected PHH, P. vivax infected and untreated PHH, P. vivax infected and treated with vehicle (DMSO) and P. vivax infected and treated with MMV048 following a time course as shown in FIG. 5. Briefly, 250 μL of highest CD9 and CD81 SEC fractions were lyophilized prior to protein digestion. Samples were processed for peptide digestion by conventional trypsin digestion at the Proteomics facilities of the Malaysia Genome Center. Samples were analyzed using a LTQ-Orbitrap Fusion Tribid mass spectrometer (Thermo Fisher Scientific, San Jose, Calif., USA) coupled to an Dionex Ultimate™ 3000 RSLCnano (Thermo Fisher Scientific). Peptides were loaded directly onto the analytical column and were separated by reversed-phase chromatography using a 15-cm column with an inner diameter of 50 m, packed with 2 m C18 particles spectrometer (Thermo Scientific, San Jose, Calif., USA). Chromatographic gradients started at 95% buffer A and 5% buffer B with a flow rate of 250 nl/min for 5 minutes and gradually increased to 40% buffer B and 60% A in 91 min and then to 40% buffer B and 60% A in 11 min. After each analysis, the column was washed for 10 min with 15% buffer A and 85% buffer B. Buffer A: 0.1% formic acid in water. Buffer B: 0.1% formic acid in 80% acetonitrile. The mass spectrometer was operated in positive ionization mode with nanospray voltage set at 2.4 kV and source temperature at 275° C. The acquisition was performed in DDA mode and full MS scans with 1 micro scans at resolution of 120,000 were used over a mass range of m/z 310-1800 with detection in the Orbitrap mass analyzer. In each cycle of DDA analysis, following each survey scan, the most intense ions above a threshold ion count of 10000 were selected for fragmentation. The number of selected precursor ions for fragmentation was determined by the “Top Speed” acquisition algorithm and a dynamic exclusion of 60 seconds. Fragment ion spectra were produced via HCD at normalized collision induced dissociation at normalized collision energy of 28% and they were acquired in the ion trap mass analyzer. AGC was set to 1.0 e2 and an isolation window of 1.6 m/z and a maximum injection time of 200 ms were used. Blank was injected in between samples to avoid sample carryover and to assure stability of the instrument.

Plasma-Derived EVs from P. vivax Infected FRG huHEP Mice
EVs samples (3 mice replicates from each experimental group, as shown in FIG. 2A), were processed for LC/MS-MS. Briefly, 100 μL of highest CD9 and CD5L SEC fractions were processed for peptide digestion using commercial kit (PreOmics) according to the manufacturers' protocol adapted for samples containing <20 μg of protein as described above. For comparison, we normalized the amount of sample to be analyzed by LC-MS/MS relative to the amount of FRG huHEP mice plasma processed for EVs isolation. Between 0.8 and 2 μg of each sample were analyzed using an LTQ-Orbitrap Fusion Lumos mass spectrometer (Thermo Fisher Scientific, San Jose, Calif., USA) coupled to an EASY-nLC 1200 (Thermo Fisher Scientific (Proxeon), Odense, Denmark). Peptides were loaded directly onto the analytical column and were separated by reversed-phase chromatography using a 50-cm column with an inner diameter of 75 m, packed with 2 m C18 particles spectrometer (Thermo Scientific, San Jose, Calif., USA). Chromatographic gradients started at 95% buffer A and 5% buffer B with a flow rate of 300 nL/min for 5 minutes and gradually increased to 25% buffer B and 75% A in 79 min and then to 40% buffer B and 60% A in 11 min. After each analysis, the column was washed for 10 min with 10% buffer A and 90% buffer B. Buffer A: 0.1% formic acid in water. Buffer B: 0.1% formic acid in 80% acetonitrile. The mass spectrometer was operated in positive ionization mode with nanospray voltage set at 2.4 kV and source temperature at 275° C. The acquisition was performed in DDA mode and full MS scans with 1 micro scans at resolution of 120,000 were used over a mass range of m/z 350-1500 with detection in the Orbitrap mass analyzer. In each cycle of DDA analysis, following each survey scan, the most intense ions above a threshold ion count of 10000 were selected for fragmentation. The number of selected precursor ions for fragmentation was determined by the “Top Speed” acquisition algorithm and a dynamic exclusion of 60 seconds. Fragment ion spectra were produced via HCD at normalized collision energy of 28% and they were acquired in the ion trap mass analyzer. AGC was set to 1E4 and an isolation window of 1.6 m/z and a maximum injection time of 200 ms were used. Digested bovine serum albumin (New England Biolabs cat #P8108S) was analyzed between each sample to avoid sample carryover and to assure stability of the instrument and QCloud has been used to control instrument longitudinal performance during the project.

### Mass Spectrometry Data Analysis

EVs from P. vivax Infected PHH

Acquired spectra were analyzed using the Proteome Discoverer software suite (v2.1, Thermo Fisher Scientific) and the Mascot search engine (v2.6, Matrix Science). The data were searched against a customized database including P. vivax (all strains: 52920 entries) and Swiss-Prot human (20581 entries) and mouse (17171 entries) databases (April 2019) plus a list of common contaminants and all the corresponding decoy entries. For peptide identification, a precursor ion mass tolerance of 10 ppm was used for MS1 level, trypsin was chosen as enzyme, and up to two missed cleavages were allowed. The fragment ion mass tolerance was set to 0.6 Da for MS2 spectra. Oxidation of methionine and N-terminal protein acetylation were used as variable modifications whereas carbamidomethylation on cysteines was set as a fixed modification. A minimum peptide length of 7 was set. FDR in peptide identification was set to a maximum of 1%. Proteins identified in PHH were classified in H. sapiens and P. vivax proteins. Human proteins identified as master proteins with high confidence were retained irrespective of the number of unique peptides. Parasite proteins identified in the group of uninfected cells were excluded as false-positives. Uniprot accession numbers of P. vivax proteins were used to retrieve protein sequences and used to identify their respective ID in Sal I and PVP01 genome through Blast analysis in PlasmoDB.

Plasma-Derived EVs from P. vivax Infected FRG huHEP Mice

Acquired spectra were analyzed using the Proteome Discoverer software suite (v2.3, Thermo Fisher Scientific) and the Mascot search engine (v2.6, Matrix Science). The data were searched against a customized database including P. vivax (all strains: 52920 entries) and Swiss-Prot human (20581 entries) and mouse (17171 entries) databases (April 2019) plus a list of common contaminants and all the corresponding decoy entries. For peptide identification, a precursor ion mass tolerance of 7 ppm was used for MS1 level, trypsin was chosen as enzyme, and up to three missed cleavages were allowed. The fragment ion mass tolerance was set to 0.5 Da for MS2 spectra. Oxidation of methionine and N-terminal protein acetylation were used as variable modifications whereas carbamidomethylation on cysteines was set as a fixed modification. A minimum peptide length of 7 was set. FDR in peptide identification was set to a maximum of 1%. Proteins identified in plasma-derived EVs were initially classified according to species H sapiens, M musculus and P. vivax. H. sapiens and M. musculus proteins identified with 2 unique peptides or more were retained. P. vivax proteins were classified according to strains. Proteins identified with 1 unique peptide, or more were retained. Parasite proteins identified in the group of uninfected mice were excluded as false positive. Uniprot accession numbers of P. vivax proteins were used to retrieve protein sequences and used to identify their respective ID in Sal I and PVP01 genome through Blast analysis in PlasmoDB. Proteins associated to EVs from each experimental group were compared quantitatively. Peptide quantification data were retrieved from the “Precursor ion area detector” node from Proteome Discoverer (v2.3) using 2 ppm mass tolerance for the peptide extracted ion current (XIC). Protein relative quantification was performed using the AEpS which is calculated as the average areas of the top 3 most abundant peptides per protein. The obtained values were used to calculate PR using a model that takes into account all peptide ratios for each of the conditions and therefore it gives the best relative protein quantification measure. For each protein ratio in each of the comparisons an adjusted p-value was given.

P. vivax Hypnozoite Biomarker Discovery
Liver Stage In Vitro P. vivax Model

First, all P. vivax proteins were selected, independently of the strain and the experimental condition. Next, the presence/absence of proteins were compared in the EVs proteome of DMSO-treated vs. MMV048 treated P. vivax PHH at each infection time point.

Liver Stage In Vivo P. vivax Model

First, all P. vivax proteins were selected, irrelevant of the strain and the EV sample. Next, intragroup PR of the three following experimental groups were compared: Group 2: MB-D8-MMV048 vs. MB-DMSO; Group 3: IV-D8- MMV048 vs. IV-D8-DMSO and Group 4: IV-D21-MMV048 vs IV-D21-DMSO. Further excluded were proteins present in Group 5: IV-TQ-D21 as false-positives. Proteins with a protein ratio >1 and statistical significance (p value<0.01) were considered. Finally, proteins found exclusively in MMV048-treated animals were selected in an intergroup comparison. In a second attempt to identify other possible candidates overlooked in the previous analysis, AEpS data and down selected proteins were explored that fulfill the previous criteria disregarding statistical significance. Expression levels of candidate proteins were retrieved from hypnozoites transcriptomic data previously published (Gural N, et al. In Vitro Culture, Drug Sensitivity, and Transcriptome of Plasmodium Vivax Hypnozoites. Cell Host Microbe 23, 395-406 e394 (2018)). Additionally, structural features (presence of transmembrane domains and signal peptides) of candidate proteins were retrieved from PlasmoDB.

Analysis of Human Proteome from Plasma-Derived EVs from P. vivax Infected FRG huHep Mice.

A statistical analysis was performed of human AEpS proteomic data from each experimental group and compared EVs protein content of P. vivax-infected DMSO-treated mice from all groups with uninfected mice to identify proteins associated to infection. In order to identify potential human biomarkers of hypnozoite infections, we performed an intragroup comparison of human proteins from MMV048-treated mice with it respective DMSO-treated control mice in groups 2, 3 and 4. In addition, we also compared DMSO-treated mice from experimental groups 2 and 3 to identify proteins associated to mosquito bite or to intravenous infection. A similar comparison was done between human proteins identified in EVs from infected DMSO-treated mice from group 3 and 4 to associate proteins to early (8 days post-infection) and late (21 days post-infection) infections. Finally, we compared human proteins from EVs from DMSO-treated mice of group 4 with mice treated with Tafenoquine in group 5 to identify proteins associated to EVs in radical cure treatment with this drug. Briefly, human proteins identified with >1 unique peptide and present in more than 3 mice were accepted for this analysis (Table 1). Protein levels were log-10 transformed to guarantee data normality. Missing data given the limit of detection were imputed by generating random samples from left truncated lognormal distribution using the R package called truncdist (Nadarajah S K, S;. R Programs for Truncated Distributions. Journal of Statistical Software 16, 1-8 (2006)). PCA was used as a quality control of replicates. Linear models were used to assess association between proteins of the above-mentioned comparisons. The obtained p-values were corrected for multiple comparisons using false discovery rate approach to avoid false positive results.

### Data Availability

EV isolation and characterization: All relevant data of the disclosed examples have been deposited to the EV-TRACK knowledgebase (EV-TRACK ID: EV200176) (Consortium E-T, et al. EV-TRACK: transparent reporting and centralizing knowledge in extracellular vesicle research. Nat Methods 14, 228-232 (2017)).

Mass spectrometry proteomic data has been deposited to the ProteomeXchange Consortium via PRIDE partner repository (Vizcaino J A, et al. 2016 update of the PRIDE database and its related tools. Nucleic Acids Res 44, 11033 (2016)) with the dataset identifier PXD023276.

While illustrative embodiments have been illustrated and described, it will be appreciated that various changes can be made therein without departing from the spirit and scope of the invention.

TABLE 1
Putative P. vivax hypnozoite biomarker
candidates identified in plasma-derived EVs from
P. vivax infected FRG huHEP mice treated with MMV048.

[symbol]

| [symbol] | [symbol] | [symbol] | [symbol] | [symbol] | [symbol] | [symbol] | [symbol] | [symbol] |
|---|---|---|---|---|---|---|---|---|
| | [symbol] | [symbol] | | [symbol] | | [symbol] | | |
| | [symbol] | | | [symbol] | | | | |
| | [symbol] | | | | | | | |
| [symbol] | [symbol] | [symbol] | [symbol] | [symbol] | [symbol] | [symbol] | [symbol] | [symbol] |
| | [symbol] | [symbol] | | [symbol] | | [symbol] | | |
| | [symbol] | [symbol] | | [symbol] | | [symbol] | | |
| | [symbol] | | | | | | | |
| | [symbol] | | | | | | | |
| [symbol] | [symbol] | [symbol] | [symbol] | [symbol] | [symbol] | [symbol] | [symbol] | [symbol] |
| | [symbol] | [symbol] | | [symbol] | | [symbol] | | |
| | [symbol] | | | [symbol] | | [symbol] | | |
| | [symbol] | | | | | | | |
| [symbol] | [symbol] | [symbol] | [symbol] | [symbol] | [symbol] | | [symbol] | [symbol] |
| | [symbol] | [symbol] | | | [symbol] | | | |
| | [symbol] | | | | | | | |
| | [symbol] | | | | | | | |
| [symbol] | [symbol] | [symbol] | [symbol] | [symbol] | [symbol] | [symbol] | [symbol] | [symbol] |
| | [symbol] | [symbol] | | [symbol] | | [symbol] | | |
| | [symbol] | | | | | | | |
| | [symbol] | | | | | | | |
| | [symbol] | | | | | | | |
| [symbol] | [symbol] | [symbol] | [symbol] | [symbol] | [symbol] | [symbol] | [symbol] | [symbol] |
| | [symbol] | [symbol] | | | | [symbol] | | |
| | [symbol] | [symbol] | | | | [symbol] | | |
| | [symbol] | | | | | | | |
| | [symbol] | | | | | | | |

| | | | | | | | |
|---|---|---|---|---|---|---|---|
| [symbol] | [symbol] | [symbol] | [symbol] | [symbol] | [symbol] | [symbol] | |
| | | | | | | [symbol] | [symbol] |
| | | | | | | [symbol] | |
| | | | | | | [symbol] | |
| | | | | | | [symbol] | [symbol] |
| [symbol] | [symbol] | [symbol] | [symbol] | [symbol] | [symbol] | [symbol] | |
| | | | | | | [symbol] | |
| | | | | | | [symbol] | [symbol] |
| | | | | | | [symbol] | |
| | | | | | | [symbol] | |
| | | | | | [symbol] | [symbol] | [symbol] |
| | | | | | [symbol] | [symbol] | |
| | | | | | [symbol] | [symbol] | |
| | | | | | | [symbol] | [symbol] |
| | | | | | | [symbol] | |
| | | | | | | [symbol] | |
| | | | | | | [symbol] | [symbol] |
| | | | | | [symbol] | [symbol] | |
| | | | | | | [symbol] | |

[symbol]

indicates data missing or illegible when filed

1. A method of detecting the presence of hypnozoite stage of Plasmodium vivax in a liver cell, the method comprising:
obtaining one or more extracellular vesicles (EVs) secreted by the liver cell; and detecting the presence of male gamete membrane fusion protein (HAP2) protein and/or proliferating cell nucleolar antigen P120 (putative; PVP01_0930100) in the one or more EVs;
wherein HAP2 protein and/or proliferating cell nucleolar antigen P120 is detected in the one or more EVs when the hypnozoite stage of P. vivax is present in the liver cell.

2. The method of claim 1, wherein obtaining one or more EVs comprises performing size exclusion chromatography (SEC) on a sample containing EVs.

3. The method of claim 2, wherein obtaining one or more EVs further comprises detecting an EV marker to confirm the presence of EVs in the sample, wherein the EV marker is optionally selected from CD9, CD81, and CD5L.

4. The method of claim 1, wherein detecting the presence of HAP2 protein or proliferating cell nucleolar antigen P120 comprises contacting the one or more EVs with an affinity reagent that binds to HAP2 or proliferating cell nucleolar antigen P120, respectively.

5-6. (canceled)

7. The method of claim 1, wherein detecting the presence of HAP2 protein or proliferating cell nucleolar antigen P120 comprises digesting proteins in EVs followed by liquid chromatograph (LC) and mass spectrometry (MS).

8-12. (canceled)

13. A method of detecting the presence of a latent Plasmodium vivax infection in a subject, the method comprising:
obtaining a liquid biological sample from the subject comprising one or more extracellular vesicles (EVs) secreted by liver cells; and
detecting the presence of male gamete membrane fusion protein (HAP2) protein and/or proliferating cell nucleolar antigen P120 (putative; PVP01_0930100) in the one or more EVs;
wherein HAP2 protein and/or proliferating cell nucleolar antigen P120 is detected in the one or more EVs when the subject has a latent P. vivax infection.

14. The method of claim 13, wherein the latent P. vivax is characterized by the presence of a hypnozoite stage of P. vivax in a liver cell in the subject.

15. The method of claim 13, further comprising isolating one or more EVs from the liquid biological sample.

16. The method of claim 15, further comprising detecting an EV marker to confirm the presence of EVs, wherein the EV marker is optionally selected from CD9, CD81, and CD5L.

17. The method of claim 15, wherein isolating one or more EV's comprises performing size exclusion chromatography (SEC) on the liquid biological sample.

18. The method of claim 13, wherein detecting the presence of HAP2 protein or proliferating cell nucleolar antigen P120 comprises contacting the one or more EVs with an affinity reagent that specifically binds to HAP2 or proliferating cell nucleolar antigen P120, respectively.

19-20. (canceled)

21. The method of claim 13, wherein detecting the presence of HAP2 protein or proliferating cell nucleolar antigen P120 comprises digesting proteins in EVs followed by liquid chromatograph (LC) and mass spectrometry (MS).

22-26. (canceled)

27. A method of diagnosing and treating a subject with a latent Plasmodium vivax infection, comprising
obtaining a biological sample from the subject with one or more extracellular vesicles (EVs) secreted by the subject's liver cells;
detecting the presence of male gamete membrane fusion protein (HAP2) protein and/or proliferating cell nucleolar antigen P120 (putative; PVP01_0930100) in the one or more EVs, wherein a detected presence of the HAP2 protein and/or proliferating cell nucleolar antigen P120 in the one or more EVs indicates a latent infection of P. vivax in the subject; and
treating the latent P. vivax infection in the subject.

28. The method of claim 27, wherein the latent P. vivax is characterized by the presence of a hypnozoite stage of P. vivax in a liver cell in the subject.

29. (canceled)

30. The method of claim 27, further comprising isolating one or more EVs from the biological sample.

31. The method of claim 27, further comprising detecting an EV marker to confirm the presence of EVs, wherein the EV marker is optionally selected from CD9, CD81, and CD5L.

32. The method of claim 31, wherein isolating one or more EVs comprises performing size exclusion chromatography (SEC) on the biological sample.

33. The method of claim 27, wherein detecting the presence of HAP2 protein or proliferating cell nucleolar antigen P120 comprises contacting the one or more EVs with an affinity reagent that specifically binds to HAP2 or proliferating cell nucleolar antigen P120, respectively.

34-35. (canceled)

36. The method of claim 27, wherein detecting the presence of HAP2 protein or proliferating cell nucleolar antigen P120 comprises digesting proteins in EVs followed by liquid chromatograph (LC) and mass spectrometry (MS).

37. (canceled)

38. The method of claim 27, wherein treating comprises administering to the subject a therapeutic amount of a composition comprising 8-aminoquinoline.

39-40. (canceled)


SEQUENCE LISTING (7 sequences)

SEQ ID NO: 1 - protein, 66 aa, Plasmodium falciparum
SNNTMNSDIKKDVSFNRIKSNRYNKENHKNKKRKTKGNHSKYSGTSMESTLTNTSPSSTPDNLSES

SEQ ID NO: 2 - protein, 64 aa, Plasmodium ovale
SSEHTMVGKNSSAYSKRLTKNRGYSQTSKKKNSKITRNRNFSRCSRSSLGSTVTYSTHSTSSVN

SEQ ID NO: 3 - protein, 69 aa, Plasmodium malariae
SNSQERVTKNSSVHTNKLTTHTGHTQTRKIGKKKNIKGTSDLGKYNGTSLESSVTYSSQSTTSVDLSSC

SEQ ID NO: 4 - protein, 80 aa, Plasmodium knowlesi
SMCQKSGDANGSFRVNKSTKSGGYKEGRRKKKKKKFGKMGNRTMPGAHSHTDFNQHCGSSSEGNVMYSSQSASSIDLSQS

SEQ ID NO: 5 - protein, 80 aa, Plasmodium vivax
SMSQKGGTRNGSFRVRKSAKSEGVKRTRRKKKKKKSGRMGNKTMTGAHSRVDYSRHGGSSSEGTAIYSSQSASSIDLSQS

SEQ ID NO: 6 - protein, 80 aa, Plasmodium vivax
SMSQKGGTRNGSFRVKKSAKSEALKQTRRKKKKKKSGRMGNKTMTGAHSRNDYSQHGGSSSEGTAIYSSQSASSIDLSQS

SEQ ID NO: 7 - protein, 80 aa, Plasmodium sp
SMSQKGSNRNGSFRVKKSTKSEGYKQSSRKKKKKKSGKIGNKTMTGAHSHTDFNQHCGSSSEGTVMYSSQSASSIDLSQS